(19) United States
(12) Patent Application Publication
(10) Pub. No.: US 2016/0271094 A1
(43) Pub. Date: Sep. 22, 2016

(54) Laquinimod Combination Therapy For Treatment Of Multiple Sclerosis

(71) Applicants: Volker Knappertz, Potomac, MD (US); Joel Kaye, Netanya (IL)

(72) Inventors: Volker Knappertz, Potomac, MD (US); Joel Kaye, Netanya (IL)

(73) Assignee: Teva Pharmaceutical Industries, Ltd., Petach-Tikva (IL)

(21) Appl. No.: 15/169,139

(22) Filed: May 31, 2016

Related U.S. Application Data
(63) Continuation of application No. 14/498,024, filed on Sep. 26, 2014.
(60) Provisional application No. 61/883,698, filed on Sep. 27, 2013.

(51) Int. Cl. A61K 31/225; A61K 45/06; A61K 31/4704

(52) U.S. Cl. CPC A61K 31/225; A61K 31/4704; A61K 45/06

(57) ABSTRACT

The subject invention provides a method for treating a subject afflicted with a form of multiple sclerosis (MS) or presenting a clinically isolated syndrome (CIS) comprising periodically administering to the subject an amount of laquinimod and an amount of a compound of formula (I):

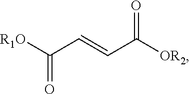

as described herein. The subject invention also provides packages and pharmaceutical compositions comprising laquinimod and a compound of formula (I) as described herein. The subject invention further provides uses of said compounds, pharmaceutical compositions and packages in treating a subject afflicted with a form of MS or presenting a CIS.

This application claims benefit of U.S. Provisional Application No. 61/883,698, filed Sep. 27, 2013, the entire content of which is hereby incorporated by reference herein.

Throughout this application, various publications are referred to by first author and year of publication. Full citations for these publications are presented in a References section immediately before the claims. Disclosures of the documents and publications referred to herein are hereby incorporated in their entireties by reference into this application.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

It is generally assumed that MS is mediated by some kind of autoimmune process possibly triggered by infection and superimposed upon a genetic predisposition. It is a chronic inflammatory condition that damages the myelin of the Central Nervous System (CNS). The pathogenesis of MS is characterized by the infiltration of autoreactive T-cells from the circulation directed against myelin antigens into the CNS (Bjartmar, 2002).

In addition to the inflammatory phase in MS, axonal loss occurs early in the course of the disease and can be extensive over time, leading to the subsequent development of progressive, permanent, neurologic impairment and, frequently, severe disability (Neuhaus, 2003). Symptoms associated with the disease include fatigue, spasticity, ataxia, weakness, bladder and bowel disturbances, sexual dysfunction, pain, tremor, paroxysmal manifestations, visual impairment, psychological problems and cognitive dysfunction (EMEA Guideline, 2006).

MS disease activity can be monitored by cranial scans, including magnetic resonance imaging (MRI) of the brain, accumulation of disability, as well as rate and severity of relapses. The diagnosis of clinically definite MS as determined by the Poser criteria (Poser, 1983) requires at least two neurological events suggesting demyelination in the CNS separated in time and in location. A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesion go on to develop MS, while approximately 20 percent have a self-limited process (Brex, 2002; Frohman, 2003).

Various MS disease stages and/or types are described in Multiple Sclerosis Therapeutics (Duntiz, 1999). Among them, relapsing remitting multiple sclerosis (RRMS) is the most common form at the time of initial diagnosis. Many subjects with RRMS have an initial relapsing-remitting course for 5-15 years, which then advances into the secondary progressive MS (SPMS) disease course.

Relapses result from inflammation and demyelination, whereas restoration of nerve conduction and remission is accompanied by resolution of inflammation, redistribution of sodium channels on demyelinated axons and remyelination (Neuhaus, 2003; Noseworthy, 2000).

In April 2001, an international panel in association with the National MS Society of America recommended diagnostic criteria for multiple sclerosis. These criteria became known as the McDonald Criteria. The McDonald Criteria make use of MRI techniques and are intended to replace the Poser Criteria and the older Schumacher Criteria (McDonald, 2001). The McDonald Criteria was revised in March 2005 by an international panel (Polman, 2005) and updated again in 2010 (Polman, 2010).

Intervention with disease-modifying therapy at relapsing stages of MS is suggested to reduce and/or prevent accumulating neurodegeneration (Hohlfeld, 2000; De Stefano, 1999). There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and Fingolimod (Gilenya®). Most of them are believed to act as immunomodulators. Mitoxantrone and natalizumab are believed to act as immunesuppressants. However, the mechanisms of action of each have been only partly elucidated. Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

Laquinimod

Laquinimod (LAQ) is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851. The mechanism of action of laquinimod is not fully understood. Animal studies show it causes a Th1 (T helper 1 cell, which produces pro-inflammatory cytokines) to Th2 (T helper 2 cell, which produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runstrom, 2006; Brück, 2011).

Combination Therapy

The administration of two drugs to treat a given condition, such as multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of GA and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of combination therapy of two drugs, in particular laquinimod and a second agent, cannot be predicted until the results of combination studies are available.

### BRIEF DESCRIPTION OF THE DRAWINGS

FIG. 1 is a graphical representation of the experimental results from Example 1B (LAQ=laquinimod).

FIG. 2 is a graphical representation of the experimental results (% of Group Mean Body Weight) from Example 1C (LQ=laquinimod).

FIG. 3 is a graphical representation of the experimental results (Group Mean Score, Mean±SE) from Example 1C (LQ=laquinimod).

### SUMMARY OF THE INVENTION

The subject invention provides a method of treating a subject afflicted with a form of multiple sclerosis (MS) or presenting a clinically isolated syndrome (CIS) comprising periodically administering to the subject a) an amount of laquinimod or a pharmaceutically acceptable salt thereof, and b) an amount of a compound of formula (I):

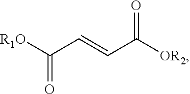

wherein R₁ is H, C₁-C₁₂ alkyl, C₂-C₁₂ alkenyl, C₂-C₁₂ alkynyl or C₃-C₈ cycloalkyl; and R₂ is H, alkyl, C₂-C₁₂ alkenyl, C₂-C₁₂ alkynyl or C₃-C₈ cycloalkyl, or a pharmaceutically acceptable salt thereof, wherein the amounts when taken together are more effective to treat the subject than when each agent at the same amount is administered alone.

The subject invention also provides a package comprising: a) a first pharmaceutical composition comprising an amount of laquinimod or a pharmaceutically acceptable salt thereof and a pharmaceutically acceptable carrier; b) a second pharmaceutical composition comprising an amount of a compound of formula (I):

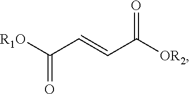

wherein R₁ is H, C₁-C₁₂ alkyl, C₂-C₁₂ alkenyl, C₂-C₁₂ alkynyl or C₃-C₈ cycloalkyl; and R₂ is H, alkyl, C₂-C₁₂ alkenyl, C₂-C₁₂ alkynyl or C₃-C₈ cycloalkyl, or a pharmaceutically acceptable salt thereof and a pharmaceutically acceptable carrier; and c) instruction for use for the first and the second pharmaceutical composition together to treat a subject afflicted with a form of MS or presenting a CIS.

The subject invention also provides a pharmaceutical composition comprising an amount of laquinimod or a pharmaceutically acceptable salt thereof and an amount of a compound of formula (I):

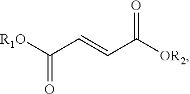

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, and at least one pharmaceutically acceptable carrier.

The subject invention also provides a pharmaceutical composition in unit dosage form, useful in treating a subject afflicted with MS or presenting a CIS, which comprises: a) an amount of laquinimod or a pharmaceutically acceptable salt thereof; b) an amount of a compound of formula (I):

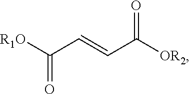

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, wherein the respective amounts of said laquinimod and said compound of formula (I) in said composition are effective, upon concomitant administration to said subject of one or more of said unit dosage forms of said composition, to treat the subject.

The subject invention also provides a pharmaceutical composition comprising an amount of laquinimod or a pharmaceutically acceptable salt thereof for use in treating a subject afflicted with MS or presenting a CIS as an add-on therapy or in combination with, or simultaneously, contemporaneously or concomitantly with a compound of formula (I):

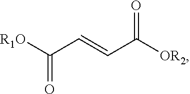

wherein R₁ is H, C₁-C₁₂ alkyl, C₂-C₁₂ alkenyl, C₂-C₁₂ alkynyl or C₃-C₈ cycloalkyl; and R₂ is H, alkyl, C₂-C₁₂ alkenyl, C₂-C₁₂ alkynyl or C₃-C₈ cycloalkyl, or a pharmaceutically acceptable salt thereof.

The subject invention also provides a pharmaceutical composition comprising an amount of a compound of formula (I):

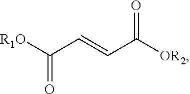

wherein R₁ is H, C₁-C₁₂ alkyl, C₂-C₁₂ alkenyl, C₂-C₁₂ alkynyl or C₃-C₈ cycloalkyl; and R₂ is H, alkyl, C₂-C₁₂ alkenyl, C₂-C₁₂ alkynyl or C₃-C₈ cycloalkyl, or a pharmaceutically acceptable salt thereof, for use treating a subject afflicted with MS or presenting a CIS as an add-on therapy or in combination with, or simultaneously, contemporaneously or concomitantly with laquinimod or a pharmaceutically acceptable salt thereof.

The subject invention also provides use of: a) an amount of laquinimod or pharmaceutically acceptable salt thereof; and b) an amount of a compound of formula (I):

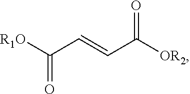

wherein R₁ is H, C₁-C₁₂ alkyl, C₂-C₁₂ alkenyl, C₂-C₁₂ alkynyl or C₃-C₈ cycloalkyl; and R₂ is H, alkyl, C₂-C₁₂ alkenyl, C₂-C₁₂ alkynyl or C₃-C₈ cycloalkyl, or pharmaceutically acceptable salt thereof, in the preparation of a combination for treating a subject afflicted with MS or presenting a CIS wherein the amount of laquinimod and the amount of the compound of formula (I) are administered simultaneously or contemporaneously.

The subject invention also provides laquinimod for use as an add-on therapy or in combination with a compound of formula (I):

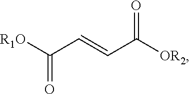

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, in treating a subject afflicted with MS or presenting a CIS.

The subject invention also provides a compound of formula (I):

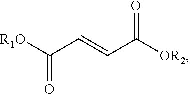

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, for use as an add-on therapy or in combination with laquinimod or a pharmaceutically acceptable salt thereof in treating a subject afflicted with MS or presenting a CIS.

The subject invention also provides laquinimod or a pharmaceutically acceptable salt thereof and a compound of formula (I):

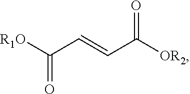

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, for the treatment of a subject afflicted with MS or presenting a CIS, wherein the laquinimod and the compound of formula (I) are administered simultaneously, separately or sequentially.

The subject invention also provides a product containing an amount of laquinimod or a pharmaceutically acceptable salt thereof and an amount of a compound of formula (I):

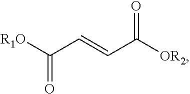

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, for simultaneous, separate or sequential use in treating a subject afflicted with MS or presenting a CIS.

The subject invention also provides a therapeutic package for dispensing to, or for use in dispensing to, a subject afflicted with MS or presenting a CIS, which comprises: a) one or more unit doses, each such unit dose comprising: i) an amount of laquinimod or a pharmaceutically acceptable salt thereof and ii) an amount a compound of formula (I):

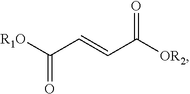

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, wherein the respective amounts of said laquinimod and said compound of formula (I) in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

### DETAILED DESCRIPTION OF THE INVENTION

The subject invention provides a method of treating a subject afflicted with a form of multiple sclerosis (MS) or presenting a clinically isolated syndrome (CIS) comprising periodically administering to the subject a) an amount of laquinimod or a pharmaceutically acceptable salt thereof, and b) an amount of a compound of formula (I):

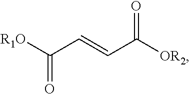

wherein R₁ is H, C₁-C₁₂ alkyl, C₂-C₁₂ alkenyl, C₂-C₁₂ alkynyl or C₃-C₈ cycloalkyl; and R₂ is H, alkyl, C₂-C₁₂ alkenyl, C₂-C₁₂ alkynyl or C₃-C₈ cycloalkyl, or a pharmaceutically acceptable salt thereof, wherein the amounts when taken together are more effective to treat the subject than when each agent at the same amount is administered alone.

In one embodiment, the MS is relapsing MS. In another embodiment, the relapsing MS is relapsing-remitting MS.

In one embodiment, the amount of laquinimod and the amount of the compound of formula (I) when taken together is effective to reduce a symptom of MS in the subject. In another embodiment, the symptom is a MRI-monitored MS disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

In one embodiment, the amount of laquinimod and the amount of the compound of formula (I) when taken together is effective to decrease or inhibit reduction of brain volume. In another embodiment, brain volume is measured by percent brain volume change (PBVC).

In one embodiment, the amount of laquinimod and the amount of the compound of formula (I) when taken together is effective to increase time to confirmed disease progression. In another embodiment, time to confirmed disease progression is increased by 20-60%.

In one embodiment, the amount of laquinimod and the amount of the compound of formula (I) when taken together is effective to decrease abnormalities observed in whole Brain MTR histogram.

In one embodiment, the accumulation of physical disability is measured by Kurtzke Expanded Disability Status Scale (EDSS) score. In another embodiment, the accumulation of physical disability is assessed by the time to confirmed disease progression as measured by Kurtzke Expanded Disability Status Scale (EDSS) score. In another embodiment, the subject had an EDSS score of 0-5.5 at baseline. In another embodiment, the subject had an EDSS score of 1.5-4.5 at baseline. In another embodiment, the subject had an EDSS score of 5.5 or greater at baseline. In another embodiment, confirmed disease progression is a 1 point increase of the EDSS score. In yet another embodiment, confirmed disease progression is a 0.5 point increase of the EDSS score.

In one embodiment, impaired mobility is assessed by the Timed-25 Foot Walk test. In another embodiment, impaired mobility is assessed by the 12-Item MS Walking Scale (MSWS-12) self-report questionnaire. In another embodiment, impaired mobility is assessed by the Ambulation Index (AI). In another embodiment, impaired mobility is assessed by the Six-Minute Walk (6MW) Test. In another embodiment, impaired mobility is assessed by the Lower Extremity Manual Muscle Test (LEMMT) Test.

In one embodiment, the amount of laquinimod and the amount of the compound of formula (I) when taken together is effective to reduce cognitive impairment. In another embodiment, cognitive impairment is assessed by the Symbol Digit Modalities Test (SDMT) score.

In one embodiment, general health status is assessed by the EuroQoL (EQ5D) questionnaire, Subject Global Impression (SGI) or Clinician Global Impression of Change (CGIC). In another embodiment, functional status is measured by the subject's Short-Form General Health survey (SF-36) Subject Reported Questionnaire score. In another embodiment, quality of life is assessed by SF-36, EQ5D, Subject Global Impression (SGI) or Clinician Global Impression of Change (CGIC). In another embodiment, the subject's SF-36 mental component summary score (MSC) is improved. In another embodiment, the subject's SF-36 physical component summary sore (PSC) is improved.

In one embodiment, fatigue is assessed by the EQ5D, the subject's Modified Fatigue Impact Scale (MFIS) score or the French valid versions of the Fatigue Impact Scale (EMIF-SEP) score. In another embodiment, symptom severity on work is measured by the work productivity and activities impairment General Health (WPAI-GH) questionnaire.

In one embodiment, laquinimod is laquinimod sodium. In another embodiment, the compound of formula (I) is a pharmaceutically acceptable salt thereof.

In an embodiment, the laquinimod and/or the compound of formula (I) is administered via oral administration. In another embodiment, the laquinimod and/or the compound of formula (I) is administered in an aerosol, an inhalable powder, an injectable, a liquid, a solid, a capsule or a tablet form. In another embodiment, the laquinimod and/or the compound of formula (I) is administered daily. In another embodiment, the laquinimod and/or the compound of formula (I) is administered more often than once daily. In yet another embodiment, the laquinimod and/or the compound of formula (I) is administered less often than once daily.

In one embodiment of the present invention, the amount laquinimod administered is less than 0.6 mg/day. In another embodiment, the amount laquinimod administered is 0.03-600 mg/day. In another embodiment, the amount laquinimod administered is 0.1-40.0 mg/day.

In another embodiment, the amount laquinimod administered is 0.1-2.5 mg/day. In another embodiment, the amount laquinimod administered is 0.25-2.0 mg/day. In another embodiment, the amount laquinimod administered is 0.5-1.2 mg/day. In another embodiment, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day. In another embodiment, the amount laquinimod administered is 1.0 mg/day. In another embodiment, the amount laquinimod administered is 1.2 mg/day. In another embodiment, the amount laquinimod administered is 1.5 mg/day. In yet another embodiment, the amount laquinimod administered is 2.0 mg/day.

In an embodiment, the amount the compound of formula (I) administered is 12-7200 mg/day. In another embodiment, the amount the compound of formula (I) administered is 120 mg/day. In another embodiment, the amount the compound of formula (I) administered is 360 mg/day. In another embodiment, the amount the compound of formula (I) administered is 480 mg/day. In another embodiment, the amount the compound of formula (I) administered is 720 mg/day.

In one embodiment of the present invention, a loading dose of laquinimod and/or the compound of formula (I) of an amount different from the intended dose is administered for a period of time at the start of the periodic administration. In another embodiment, the loading dose is double the amount of the intended dose. In yet another embodiment, the loading dose is half the amount of the intended dose.

In one embodiment, the subject is receiving laquinimod therapy prior to initiating the compound of formula (I) therapy. In another embodiment, the administration of laquinimod substantially precedes the administration of the compound of formula (I). In another embodiment, the subject is receiving the compound of formula (I) therapy prior to initiating laquinimod therapy. In another embodiment, the administration of the compound of formula (I) substantially precedes the administration of laquinimod. In another embodiment, the subject is receiving the compound of formula (I) therapy for at least 8 weeks prior to initiating laquinimod therapy. In another embodiment, the subject is receiving the compound of formula (I) therapy for at least 10 weeks prior to initiating laquinimod therapy. In another embodiment, the subject is receiving the compound of formula (I) therapy for at least 24 weeks prior to initiating laquinimod therapy. In another embodiment, the subject is receiving the compound of formula (I) therapy for at least 28 weeks prior to initiating laquinimod therapy. In another embodiment, the subject is receiving the compound of formula (I) therapy for at least 48 weeks prior to initiating laquinimod therapy. In yet another embodiment, the subject is receiving the compound of formula (I) therapy for at least 52 weeks prior to initiating laquinimod therapy.

In one embodiment, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

In another embodiment, the periodic administration of laquinimod and the compound of formula (I) continues for at least 3 days. In another embodiment, the periodic administration of laquinimod and the compound of formula (I) continues for more than 30 days. In another embodiment, the periodic administration of laquinimod and the compound of formula (I) continues for more than 42 days. In another embodiment, the periodic administration of laquinimod and the compound of formula (I) continues for 8 weeks or more. In another embodiment, the periodic administration of laquinimod and the compound of formula (I) continues for at least 12 weeks. In another embodiment, the periodic administration of laquinimod and the compound of formula (I) continues for at least 24 weeks. In another embodiment, the periodic administration of laquinimod and the compound of formula (I) continues for more than 24 weeks. In yet another embodiment, the periodic administration of laquinimod and the compound of formula (I) continues for 6 months or more.

In one embodiment, each of the amount of laquinimod when taken alone, and the amount of the compound of formula (I) when taken alone is effective to treat the subject. In another embodiment, either the amount of laquinimod when taken alone, the amount of the compound of formula (I) when taken alone, or each such amount when taken alone is not effective to treat the subject. In yet another embodiment, the subject is a human patient.

The subject invention also provides a package comprising: a) a first pharmaceutical composition comprising an amount of laquinimod or a pharmaceutically acceptable salt thereof and a pharmaceutically acceptable carrier; b) a second pharmaceutical composition comprising an amount of a compound of formula (I):

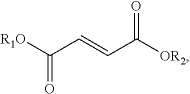

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof and a pharmaceutically acceptable carrier; and c) instruction for use for the first and the second pharmaceutical composition together to treat a subject afflicted with a form of MS or presenting a CIS.

In one embodiment, the first pharmaceutical composition, the second pharmaceutical composition, or both the first and the second pharmaceutical composition are in an aerosol, an inhalable powder, an injectable, a liquid, a solid, a capsule or a tablet form. In another embodiment, the first pharmaceutical composition, the second pharmaceutical composition, or both the first and the second pharmaceutical composition are in a liquid or a solid form. In another embodiment, the first pharmaceutical composition, the second pharmaceutical composition, or both the first and the second pharmaceutical composition are in capsule form or in tablet form.

In one embodiment, the tablets are coated with a coating which inhibits oxygen from contacting the core. In another embodiment, the coating comprises a cellulosic polymer, a detackifier, a gloss enhancer, or pigment. In another embodiment, the first pharmaceutical composition further comprises mannitol. In another embodiment, the first pharmaceutical composition further comprises an alkalinizing agent. In another embodiment, the alkalinizing agent is meglumine. In another embodiment, the first pharmaceutical composition further comprises an oxidation reducing agent.

In an embodiment, the first pharmaceutical composition is stable and free of an alkalinizing agent or an oxidation reducing agent.

In another embodiment, the first pharmaceutical composition is free of an alkalinizing agent and free of an oxidation reducing agent.

In one embodiment, the first pharmaceutical composition is stable and free of disintegrant. In another embodiment, the first pharmaceutical composition further comprises a lubricant. In another embodiment, the lubricant is present in the composition as solid particles. In another embodiment, the lubricant is sodium stearyl fumarate or magnesium stearate.

In an embodiment, the first pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In an embodiment, the package further comprises a desiccant. In another embodiment, the desiccant is silica gel.

In one embodiment, the first pharmaceutical composition is stable and has a moisture content of no more than 4%. In another embodiment, laquinimod is present in the composition as solid particles. In another embodiment, the package is a sealed packaging having a moisture permeability of not more than 15 mg/day per liter. In another embodiment, the sealed package is a blister pack in which the maximum moisture permeability is no more than 0.005 mg/day. In another embodiment, the sealed package is a bottle. In another embodiment, the bottle is closed with a heat induction liner. In another embodiment, the sealed package comprises an HDPE bottle. In another embodiment, the sealed package comprises an oxygen absorbing agent. In another embodiment, the oxygen absorbing agent is iron.

In one embodiment, the amount of laquinimod in the first composition is less than 0.6 mg. In another embodiment, the amount of laquinimod is 0.1-40.0 mg. In another embodiment, the amount of laquinimod is 0.1-2.5 mg. In another embodiment, the amount of laquinimod is 0.25-2.0 mg. In another embodiment, the amount of laquinimod is 0.5-1.2 mg. In another embodiment, the amount of laquinimod is 0.25 mg. In another embodiment, the amount of laquinimod is 0.3 mg. In another embodiment, the amount of laquinimod is 0.5 mg. In another embodiment, the amount of laquinimod is 0.6 mg. In another embodiment, the amount of laquinimod is 1.0 mg. In another embodiment, the amount of laquinimod is 1.2 mg. In another embodiment, the amount of laquinimod is 1.5 mg. In yet another embodiment, the amount of laquinimod is 2.0 mg.

In an embodiment, the amount of the compound of formula (I) is 12-7200 mg. In another embodiment, the amount of the compound of formula (I) is 120 mg. In another embodiment, the amount of the compound of formula (I) is 360 mg. In another embodiment, the amount of the compound of formula (I) is 480 mg. In another embodiment, the amount of the compound of formula (I) is 720 mg.

In one embodiment, the amount of laquinimod and the amount of the compound of formula (I) are prepared to be administered simultaneously, contemporaneously or concomitantly. In another embodiment, the package is for use in treating a subject afflicted with a form of MS or presenting a CIS.

The subject invention also provides a pharmaceutical composition comprising an amount of laquinimod or a pharmaceutically acceptable salt thereof and an amount of a compound of formula (I):

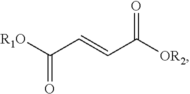

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, and at least one pharmaceutically acceptable carrier. In one embodiment, the pharmaceutical composition is for use in treating a subject afflicted with MS or presenting a CIS, wherein the laquinimod and the compound of formula (I) are prepared to be administered simultaneously, contemporaneously or concomitantly.

In an embodiment, laquinimod is laquinimod sodium. In another embodiment, the compound of formula (I) is a pharmaceutically acceptable salt thereof.

In one embodiment, the pharmaceutical composition is in an aerosol, an inhalable powder, an injectable, a liquid, a solid, a capsule or a tablet form. In another embodiment, the pharmaceutical composition is in a liquid or a solid form. In yet another embodiment, the pharmaceutical composition is in capsule form or in tablet form.

In one embodiment, the tablets are coated with a coating which inhibits oxygen from contacting the core. In another embodiment, the coating comprises a cellulosic polymer, a detackifier, a gloss enhancer, or pigment.

In one embodiment, the pharmaceutical composition further comprises mannitol. In another embodiment, the pharmaceutical composition further comprises an alkalinizing agent. In another embodiment, the alkalinizing agent is meglumine. In another embodiment, the pharmaceutical composition further comprises an oxidation reducing agent.

In one embodiment, the pharmaceutical composition is free of an alkalinizing agent or an oxidation reducing agent. In another embodiment, the pharmaceutical composition is free of an alkalinizing agent and free of an oxidation reducing agent.

In one embodiment, the pharmaceutical composition is stable and free of disintegrant. In another embodiment, the pharmaceutical composition further comprises a lubricant. In one embodiment, the lubricant is present in the composition as solid particles. In another embodiment, the lubricant is sodium stearyl fumarate or magnesium stearate.

In one embodiment, the pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In yet another embodiment, the filler is mannitol or lactose monohydrate.

In one embodiment, the amount of laquinimod in the composition is less than 0.6 mg. In another embodiment, the amount of laquinimod in the composition is 0.03-600 mg. In another embodiment, the amount of laquinimod is 0.1-40.0 mg. In another embodiment, the amount of laquinimod is 0.1-2.5 mg. In another embodiment, the amount of laquinimod is 0.25-2.0 mg. In another embodiment, the amount of laquinimod is 0.5-1.2 mg. In another embodiment, the amount of laquinimod is 0.25 mg. In another embodiment, the amount of laquinimod is 0.3 mg. In another embodiment, the amount of laquinimod is 0.5 mg. In another embodiment, the amount of laquinimod is 0.6 mg. In another embodiment, the amount of laquinimod is 1.0 mg. In another embodiment, the amount of laquinimod is 1.2 mg. In another embodiment, the amount of laquinimod is 1.5 mg. In yet another embodiment, the amount of laquinimod is 2.0 mg.

In one embodiment, the amount of the compound of formula (I) is 12-7200 mg. In another embodiment, the amount of the compound of formula (I) is 120 mg. In another embodiment, the amount of the compound of formula (I) is 240 mg. In another embodiment, the amount of the compound of formula (I) is 480 mg. In yet another embodiment, the amount of the compound of formula (I) is 720 mg.

The subject invention also provides a pharmaceutical composition in unit dosage form, useful in treating a subject afflicted with MS or presenting a CIS, which comprises: a) an amount of laquinimod or a pharmaceutically acceptable salt thereof; b) an amount of a compound of formula (I):

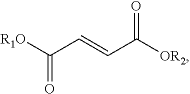

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, wherein the respective amounts of said laquinimod and said compound of formula (I) in said composition are effective, upon concomitant administration to said subject of one or more of said unit dosage forms of said composition, to treat the subject. In one embodiment, the respective amounts of said laquinimod and said compound of formula (I) in said unit dose when taken together is more effective to treat the subject than when compared to the administration of said laquinimod in the absence of said compound of formula (I) or the administration of said compound of formula (I) in the absence of said laquinimod.

The subject invention also provides a pharmaceutical composition comprising an amount of laquinimod or a pharmaceutically acceptable salt thereof for use in treating a subject afflicted with MS or presenting a CIS as an add-on therapy or in combination with, or simultaneously, contemporaneously or concomitantly with a compound of formula (I):

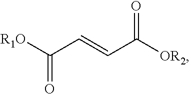

wherein R₁ is H, C₁-C₁₂ alkyl, C₂-C₁₂ alkenyl, C₂-C₁₂ alkynyl or C₃-C₈ cycloalkyl; and R₂ is H, alkyl, C₂-C₁₂ alkenyl, C₂-C₁₂ alkynyl or C₃-C₈ cycloalkyl, or a pharmaceutically acceptable salt thereof.

The subject invention also provides a pharmaceutical composition comprising an amount of a compound of formula (I):

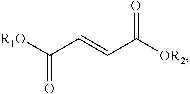

wherein R₁ is H, C₁-C₁₂ alkyl, C₂-C₁₂ alkenyl, C₂-C₁₂ alkynyl or C₃-C₈ cycloalkyl; and R₂ is H, alkyl, C₂-C₁₂ alkenyl, C₂-C₁₂ alkynyl or C₃-C₈ cycloalkyl, or a pharmaceutically acceptable salt thereof, for use treating a subject afflicted with MS or presenting a CIS as an add-on therapy or in combination with, or simultaneously, contemporaneously or concomitantly with laquinimod or a pharmaceutically acceptable salt thereof.

The subject invention also provides use of: a) an amount of laquinimod or pharmaceutically acceptable salt thereof; and b) an amount of a compound of formula (I):

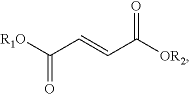

wherein R₁ is H, C₁-C₁₂ alkyl, C₂-C₁₂ alkenyl, C₂-C₁₂ alkynyl or C₃-C₈ cycloalkyl; and R₂ is H, alkyl, C₂-C₁₂ alkenyl, C₂-C₁₂ alkynyl or C₃-C₈ cycloalkyl, or pharmaceutically acceptable salt thereof, in the preparation of a combination for treating a subject afflicted with MS or presenting a CIS wherein the amount of laquinimod and the amount of the compound of formula (I) are administered simultaneously or contemporaneously.

The subject invention also provides laquinimod for use as an add-on therapy or in combination with a compound of formula (I):

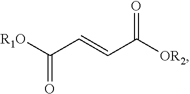

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, in treating a subject afflicted with MS or presenting a CIS.

The subject invention also provides a compound of formula (I):

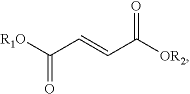

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, for use as an add-on therapy or in combination with laquinimod or a pharmaceutically acceptable salt thereof in treating a subject afflicted with MS or presenting a CIS.

The subject invention also provides laquinimod or a pharmaceutically acceptable salt thereof and a compound of formula (I):

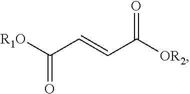

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, for the treatment of a subject afflicted with MS or presenting a CIS, wherein the laquinimod and the compound of formula (I) are administered simultaneously, separately or sequentially.

The subject invention also provides a product containing an amount of laquinimod or a pharmaceutically acceptable salt thereof and an amount of a compound of formula (I):

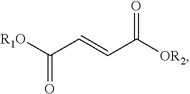

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, for simultaneous, separate or sequential use in treating a subject afflicted with MS or presenting a CIS.

The subject invention also provides a therapeutic package for dispensing to, or for use in dispensing to, a subject afflicted with MS or presenting a CIS, which comprises: a) one or more unit doses, each such unit dose comprising: i) an amount of laquinimod or a pharmaceutically acceptable salt thereof and ii) an amount a compound of formula (I):

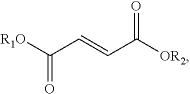

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, wherein the respective amounts of said laquinimod and said compound of formula (I) in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject. In one embodiment, the respective amounts of said laquinimod and said compound of formula (I) in said unit dose when taken together is more effective to treat the subject than when compared to the administration of said laquinimod in the absence of said compound of formula (I) or the administration of said compound of formula (I) in the absence of said laquinimod.

In an embodiment of the present invention, in the compound of formula (I): R₁ is H, C₁-C₁₂ alkyl, C₂-C₁₂ alkenyl, C₂-C₁₂ alkynyl or C₃-C₈ cycloalkyl; and R₂ is H, C₂-C₁₂ alkyl, C₂-C₁₂ alkenyl, C₂-C₁₂ alkynyl or C₃-C₈ cycloalkyl. In another embodiment, when R₁ or R₂ is H, then the other of R₁ or R₂ is other than H, or a pharmaceutically acceptable salt thereof. In another embodiment, R₁ and R₂ are the same. In another embodiment, R₁ and R₂ are different. In another embodiment, R₂ is H; and R₁ is CH₃. In another embodiment, R₁ is CH₃; and R₂ is CH₂CH₃; R₁ is CH₃; and R₂ is CH₂CH₂CH₃; R₁ is CH₃; and R₂ is CH₂CH₂CH₂CH₃; R₁ is CH₃; and R₂ is CH₂CH₂CH₂CH₂CH₃; or R₁ is CH₃; and R₂ is CH₂CH₂CH₂CH₂CH₂CH₃. In another embodiment, In another embodiment, the compound of formula (I) has the structure:

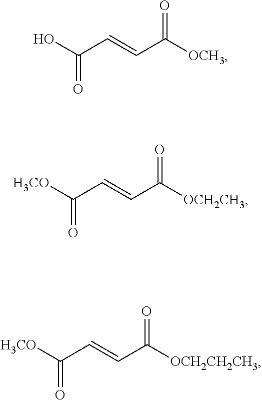

or a pharmaceutically acceptable salt thereof.

In an embodiment of the present invention, in the compound of formula (I): R₁ is H, C₂-C₁₂ alkyl, C₂-C₁₂ alkenyl, C₂-C₁₂ alkynyl or C₃-C₈ cycloalkyl; and R₂ is H, C₂-C₁₂ alkyl, C₂-C₁₂ alkenyl, C₂-C₁₂ alkynyl or C₃-C₈ cycloalkyl. In another embodiment of the present invention, in the compound of formula (I): R₁ is H, C₃-C₁₂ alkyl, C₂-C₁₂ alkenyl, C₂-C₁₂ alkynyl or C₃-C₈ cycloalkyl; and R₂ is H, C₃-C₁₂ alkyl, C₂-C₁₂ alkenyl, C₂-C₁₂ alkynyl; or C₃-C₈ cycloalkyl.

In another embodiment of the present invention, in the compound of formula (I), when one of R₁ or R₂ is H, then the other of R₁ or R₂ is other than H, or a pharmaceutically acceptable salt thereof. In another embodiment of the present invention, in the compound of formula (I), when one of R₁ or R₂ is CH₃, then the other of R₁ or R₂ is other than CH₃. In another embodiment of the present invention, in the compound of formula (I), when one of R₁ or R₂ is H, then the other of R₁ or R₂ is other than H; and when one of R₁ or R₂ is CH₃, then the other of R₁ or R₂ is other than CH₃.

In another embodiment of the present invention, in the compound of formula (I), R₁ and R₂ are the same. In another embodiment of the present invention, in the compound of formula (I), R₁ and R₂ are different.

In another embodiment of the present invention, in the compound of formula (I): R₁ is H; and R₂ is H; R₁ is H; and R₂ is CH₃; R₁ is H; and R₂ is CH₂CH₃; R₁ is H; and R₂ is CH₂CH₂CH₃; R₁ is H; and R₂ is CH₂CH₂CH₂CH₃; R₁ is H; and R₂ is CH₂CH₂CH₂CH₂CH₃; or R₁ is H; and R₂ is CH₂CH₂CH₂CH₂CH₂CH₃.

In another embodiment of the present invention, in the compound of formula (I): R₁ is CH₃; and R₂ is CH₂CH₃; R₁ is CH₃; and R₂ is CH₂CH₂CH₃; R₁ is CH₃; and R₂ is CH₂CH₂CH₂CH₃; R₁ is CH₃; and R₂ is CH₂CH₂CH₂CH₂CH₃; or R₁ is CH₃; and R₂ is CH₂CH₂CH₂CH₂CH₂CH₃.

In another embodiment of the present invention, in the compound of formula (I): R₁ is CH₂CH₃; and R₂ is CH₂CH₃; R₁ is CH₂CH₃; and R₂ is CH₂CH₂CH₃; R₁ is CH₂CH₃; and R₂ is CH₂CH₂CH₂CH₃; R₁ is CH₂CH₃; and R₂ is CH₂CH₂CH₂CH₂CH₃; or R₁ is CH₂CH₃; and R₂ is CH₂CH₂CH₂CH₂CH₂CH₃.

In another embodiment of the present invention, in the compound of formula (I): R₁ is CH₂CH₂CH₃; and R₂ is CH₂CH₂CH₃; R₁ is CH₂CH₂CH₃; and R₂ is CH₂CH₂CH₂CH₃; R₁ is CH₂CH₂CH₃; and R₂ is CH₂CH₂CH₂CH₂CH₃; or R₁ is CH₂CH₂CH₃; and R₂ is CH₂CH₂CH₂CH₂CH₂CH₃.

In one embodiment of the present invention, the compound of formula (I) has the structure:

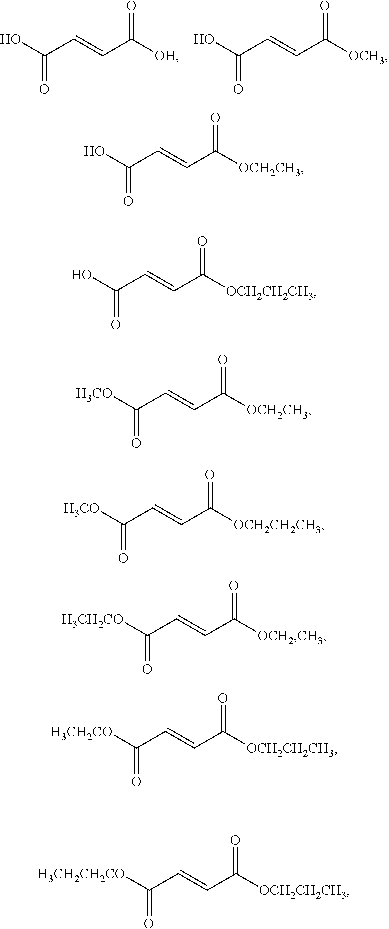

or a pharmaceutically acceptable salt thereof.

In an embodiment of the present invention, in the compound of formula (I), R₁ is H and R₂ is CH₃, or R₂ is H and R₁ is CH₃. In another embodiment of the present invention, in the compound of formula (I), R₁ is H and R₂ is CH₂CH₃. In another embodiment of the present invention, in the compound of formula (I), R₁ is CH₃ and R₂ is CH₃. In another embodiment of the present invention, in the compound of formula (I), R₁ is CH₃ and R₂ is CH₂CH₃. In another embodiment of the present invention, the compound of formula (I) is formoterol fumarate.

For the foregoing embodiments, each embodiment disclosed herein is contemplated as being applicable to each of the other disclosed embodiments. For instance, the elements recited in the method embodiments can be used in the pharmaceutical composition, package, product and use embodiments described herein and vice versa.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit may be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248.

General techniques and compositions for making dosage forms useful in the present invention are described-in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject, e.g., human patient, afflicted with multiple sclerosis, e.g., relapsing MS or presenting CIS using laquinimod with a compound of formula (I):

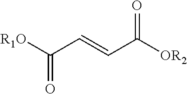

wherein R₁ is H, C₁-C₁₂ alkyl, C₂-C₁₂ alkenyl, C₂-C₁₂ alkynyl or C₃-C₈ cycloalkyl; and R₂ is H, C₁-C₁₂ alkyl, C₂-C₁₂ alkenyl, C₂-C₁₂ alkynyl or C₃-C₈ cycloalkyl, or a pharmaceutically acceptable salt thereof, which provides a more efficacious treatment than each agent alone. The use of laquinimod for multiple sclerosis had been previously suggested in, e.g., U.S. Pat. No. 6,077,851. However, the inventors have surprisingly found that the combination therapy provided herein is particularly effective for the treatment of relapsing multiple sclerosis as compared to each agent alone.

Terms

As used herein, and unless stated otherwise, each of the following terms shall have the definition set forth below.

As used herein, “laquinimod” means laquinimod acid or a pharmaceutically acceptable salt thereof.

Similarly, a “compound of formula (I)” means a compound of having the structure

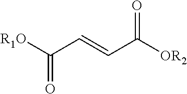

or a pharmaceutically acceptable salt thereof. In some embodiments, one or both of the R groups in is Hydrogen. In another embodiment, one or both of the hydrogen are replaced with another element or group, e.g., CH₃, CH₂CH₃ and so on. In an embodiment, the compound has the following structure:

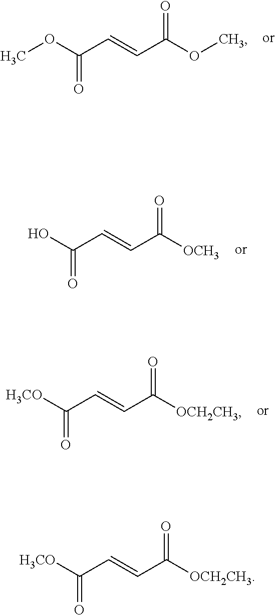

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, n−1 or n carbons in a linear or branched arrangement, and specifically includes, but not limited to, methyl, ethyl, propyl, butyl, pentyl, hexyl, heptyl, octyl, isopropyl, isobutyl, sec-butyl, t-butyl and 2-ehyl hexyl. An embodiment can be C₁-C₁₂ alkyl, C₂-C₁₂ alkyl, C₃-C₁₂ alkyl, C₄-C₁₂ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include, but are not limited to, ethenyl, propenyl, butenyl, cyclohexenyl, vinyl, and allyl. As described above with respect to alkyl, the straight, branched or cyclic portion of the alkenyl group may contain double bonds and may be substituted if a substituted alkenyl group is indicated. An embodiment can be C₂-C₁₂ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl.

As sued herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

In the compounds of the present invention, the alkyl, alkenyl, alkynyl and cycloalkyl substituents may be unsubstituted or unsubstituted, unless specifically defined otherwise. For example, a C₁-C₁₂ alkyl may be substituted with one or more substituents selected from, but are not limited to, OH, oxo, halogen, methoxy, alkoxy, nitro, cyano, or amino. The alkyl, alkenyl, alkynyl and cycloalkyl groups can be substituted by replacing one or more hydrogen atoms by non-hydrogen groups described herein to the extent possible. These include, but are not limited to, OH, oxo, halogen, alkoxy, nitro, cyano. Examples of substituted alkyl groups include, but are not limited to, hyrdoxymethyl, 2-hydroxyethyl, 2-hydroxypropyl, 3-hydroxypropyl, methoxymethyl, 2-methoxyethyl, 2-methoxypropyl, and 3-methoxypropyl.

A “salt thereof” is a salt of the instant compounds which have been modified by making acid or base salts of the compounds. The term “pharmaceutically acceptable salt” in this respect, refers to the relatively non-toxic, inorganic and organic acid or base addition salts of compounds of the present invention. For example, one means of preparing such a salt is by treating a compound of the present invention with an inorganic base. Examples of a pharmaceutically acceptable salt of compound of formula (I) include, e.g., acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, ammonium salts, alkali metal salts, sodium, zinc, potassium, calcium and magnesium salts, salts with organic bases, such as dicyclohexylamine salts, N-methyl-D-glucamine, and salts with amino acids such as arginine, lysine.

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion. Similarly, “amount” or “dose” of a compound of formula (I) as measured in milligrams refers to the milligrams of the compound present in regardless of the form of the preparation.

As used herein, a “unit dose”, “unit doses” and “unit dosage form(s)” mean a single drug administration entity/entities.

As used herein, “about” in the context of a numerical value or range means±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process. For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound selected from the group consisting of tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein also refers to Flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound selected from the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound selected from the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), des feral from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the laquinimod and a compound of formula (I). In this case, the combination may be the admixture or separate containers of the laquinimod and the compound of formula (I) that are combined just prior to administration. Contemporaneous administration refers to the separate administration of the laquinimod and the compound of formula (I) at the same time, or at times sufficiently close together that a synergistic activity relative to the activity of either the laquinimod or the compound alone is observed.

As used herein, “concomitant administration” or administering “concomitantly” means the administration of two agents given in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding laquinimod therapy to a patient already receiving therapy using compound of formula (I), or adding compound of formula (I) therapy to a patient already receiving laquinimod therapy.

As used herein, “effective” when referring to an amount of laquinimod and/or a compound of formula (I) refers to the quantity of laquinimod and/or the compound that is sufficient to yield a desired therapeutic response. Efficacy can be measured by an improvement of a symptom of multiple sclerosis. Such symptoms can include a MRI-monitored multiple sclerosis disease activity, relapse rate, accumulation of physical disability, frequency of relapses, time to confirmed disease progression, time to confirmed relapse, frequency of clinical exacerbation, brain atrophy, neuronal dysfunction, neuronal injury, neuronal degeneration, neuronal apoptosis, risk for confirmed progression, visual function, fatigue, impaired mobility, cognitive impairment, brain volume, abnormalities observed in whole Brain MTR histogram, general health status, functional status, quality of life, and/or symptom severity on work.

In an embodiment, an effective amount or regimen is an amount that is sufficient to reduce relapse rate, preserve brain tissue, decrease or inhibit reduction of brain volume (optionally brain volume is measured by percent brain volume change (PBVC)), increase time to confirmed disease progression (e.g., by 20-60% or at least 50%), reduce disability progression, decrease abnormalities observed in whole Brain MTR histogram, decrease the accumulation of physical disability (optionally measured by Kurtzke Expanded Disability Status Scale (EDSS) score, e.g., wherein the accumulation of physical disability is assessed by the time to confirmed disease progression as measured by Kurtzke Expanded Disability Status Scale (EDSS) score), improve impaired mobility (optionally assessed by the Timed-25 Foot Walk test, the 12-Item Multiple Sclerosis Walking Scale (MSWS-12) self-report questionnaire, the Ambulation Index (AI), the Six-Minute Walk (6MW) Test, or the Lower Extremity Manual Muscle Test (LEMMT) Test), reduce cognitive impairment (optionally assessed by the Symbol Digit Modalities Test (SDMT) score), improve general health (optionally assessed by the EuroQoL (EQ5D) questionnaire, Subject Global Impression (SGI) or Clinician Global Impression of Change (CGIC)), improve functional status (optionally measured by the subject's Short-Form General Health survey (SF-36) Subject Reported Questionnaire score), improve quality of life (optionally assessed by SF-36, EQ5D, Subject Global Impression (SGI) or Clinician Global Impression of Change (CGIC)), improve the subject's SF-36 mental component summary score (MSC) and/or SF-36 physical component summary sore (PSC), reduce level of fatigue (optionally assessed by the EQ5D, the subject's Modified Fatigue Impact Scale (MFIS) score or the French valid versions of the Fatigue Impact Scale (EMIF-SEP) score), or improve symptom severity on work (optionally measured by the work productivity and activities impairment General Health (WPAI-GH) questionnaire).

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

“Treating” as used herein encompasses, e.g., inducing inhibition, regression, or stasis of a disease or disorder, e.g., Relapsing MS (RMS), or alleviating, lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with MS or RMS includes any clinical or laboratory manifestation associated with MS or RMS and is not limited to what the subject can feel or observe.

As used herein, “a subject afflicted with multiple sclerosis” or “a subject afflicted with relapsing multiple sclerosis” means a subject who has been clinically diagnosed to have multiple sclerosis or relapsing multiple sclerosis (RMS), which includes relapsing-remitting multiple sclerosis (RRMS) and Secondary Progressive multiple sclerosis (SPMS).

As used herein, a subject at “baseline” is as subject prior to administration of laquinimod or a compound of formula (I) in a combination or add-on therapy as described herein.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4⁺ T cells, CD8⁺ T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

“Relapse Rate” is the number of confirmed relapses per unit time. “Annualized relapse rate” is the mean value of the number of confirmed relapses of each patient multiplied by 365 and divided by the number of days that patient is on the study drug.

“Expanded Disability Status Scale” or “EDSS” is a rating system that is frequently used for classifying and standardizing the condition of people with multiple sclerosis. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS) (Kurtzke JF, 1983).

A “confirmed progression” of EDSS, or “confirmed disease progression” as measured by EDSS score is defined as a 1 point increase from baseline EDSS if baseline EDSS was between 0 and 5.0, or a 0.5 point increase if baseline EDSS was 5.5. In order to be considered a confirmed progression, the change (either 1 point or 0.5 points) must be sustained for at least 3 months. In addition, confirmation of progression cannot be made during a relapse.

“Adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product.

“Gd-enhancing lesion” refers to lesions that result from a breakdown of the blood-brain barrier, which appear in contrast studies using gandolinium contrast agents. Gandolinium enhancement provides information as to the age of a lesion, as Gd-enhancing lesions typically occur within a six week period of lesion formation.

“Magnetization Transfer Imaging” or “MTI” is based on the magnetization interaction (through dipolar and/or chemical exchange) between bulk water protons and macromolecular protons. By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. “MT” or “Magnetization Transfer” refers to the transfer of longitudinal magnetization from the hydrogen nuclei of water that have restricted motion to the hydrogen nuclei of water that moves with many degrees of freedom.

With MTI, the presence or absence of macromolecules (e.g. in membranes or brain tissue) can be seen (Mehta, 1996; Grossman, 1994).

“Magnetization Resonance Spectroscopy” or “MRS” is a specialized technique associated with magnetic resonance imaging (MRI). MRS is used to measure the levels of different metabolites in body tissues. The MR signal produces a spectrum of resonances that correspond to different molecular arrangements of the isotope being “excited”. This signature is used to diagnose certain metabolic disorders, especially those affecting the brain, (Rosen, 2007) as well as to provide information on tumor metabolism (Golder, 2007).

As used herein “mobility” refers to any ability relating to walking, walking speed, gait, strength of leg muscles, leg function and the ability to move with or without assistance. Mobility can be evaluated by one or more of several tests including but not limited to Ambulation Index, Time 25 foot walk, Six-Minute Walk (6MW), Lower Extremity Manual Muscle Test (LEMMT) and EDSS. Mobility can also be reported by the subject, for example by questionnaires, including but not limited to 12-Item Multiple Sclerosis Walking Scale (MSWS-12). Impaired Mobility refers to any impairment, difficulty or disability relating to mobility.

“T1-weighted MRI image” refers to an MR-image that emphasizes T1 contrast by which lesions may be visualized. Abnormal areas in a T1-weighted MRI image are “hypointense” and appear as dark spots. These spots are generally older lesions.

“T2-weighted MRI image” refers to an MR-image that emphasizes T2 contrast by which lesions may be visualized. T2 lesions represent new inflammatory activity.

The “Six-Minute Walk (6MW) Test” is a commonly used test developed to assess exercise capacity in patients with COPD (Guyatt, 1985). It has been used also to measure mobility in multiple sclerosis patients (Clinical Trials Website).

The “Timed-25 Foot Walk” or “T25-FW” is a quantitative mobility and leg function performance test based on a timed 25-walk. The patient is directed to one end of a clearly marked 25-foot course and is instructed to walk 25 feet as quickly as possible, but safely. The time is calculated from the initiation of the instruction to start and ends when the patient has reached the 25-foot mark. The task is immediately administered again by having the patient walk back the same distance. Patients may use assistive devices when doing this task. The score for the T25-FW is the average of the two completed trials. This score can be used individually or used as part of the MSFC composite score (National MS Society Website).

One of the central symptoms of multiple sclerosis is fatigue. Fatigue can be measured by several tests including but not limited to decrease of French valid versions of the Fatigue Impact Scale (EMIF-SEP) score, and European Quality of Life (EuroQoL) Questionnaire (EQ5D). Other tests, including but not limited to Clinician Global Impression of Change (CGIC) and Subject Global Impression (SGI), as well as EQ-5D, can be used to evaluate the general health status and quality of life of MS patients.

“Ambulation Index” or “AI” is a rating scale developed by Hauser et al. to assess mobility by evaluating the time and degree of assistance required to walk 25 feet. Scores range from 0 (asymptomatic and fully active) to 10 (bedridden). The patient is asked to walk a marked 25-foot course as quickly and safely as possible. The examiner records the time and type of assistance (e.g., cane, walker, crutches) needed. (Hauser, 1983)

“EQ-5D” is a standardized questionnaire instrument for use as a measure of health outcome applicable to a range of health conditions and treatments. It provides a simple descriptive profile and a single index value for health status that can be used in the clinical and economic evaluation of health care as well as population health surveys. EQ-5D was developed by the “EuroQoL” Group which comprises a network of international, multilingual, multidisciplinary researchers, originally from seven centers in England, Finland, the Netherlands, Norway and Sweden. The EQ-5D questionnaire is in the public domain and can be obtained from EuroQoL.

“SF-36” is a multi-purpose, short-form health survey with 36 questions which yields an 8-scale profile of functional health and well-being scores as well as psychometrically-based physical and mental health summary measures and a preference-based health utility index. It is a generic measure, as opposed to one that targets a specific age, disease, or treatment group. The survey is developed by and can be obtained from QualityMetric, Inc. of Providence, R.I.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

Experimental Details

Since the mechanisms of action of laquinimod and compounds of formula (I), e.g., monomethyl fumarate (MMF), dimethyl fumarate (DMF), monoethyl fumarate (MEF) or ethyl methyl fumarate (EMF) have not been fully elucidated, the effect of the combined therapy cannot be predicted and must be evaluated experimentally.

### Example 1A

### Assessment of Efficacy of Laquinimod Alone or in Combination with DMF in MOG-Induced EAE

In this experiment, MOG-induced EAE Mice are treated with two doses of laquinimod (0.06 and 0.12 mg/kg) alone or with add on DMF (25 or 50 mg/kg) to assess the efficacy of laquinimod alone or in combination with DMF. MOG-induced Experimental Autoimmune Encephalomyelitis (EAE) in the C57BL/6 strain of mice is an established EAE model to test the efficacy of the candidate molecule for MS treatment.

Procedure

Disease is induced in all mice by the injection of the encephalitogenic emulsion (MOG/CFA) and intraperitoneal injection of pertussis toxin on the first day and 48 hours later.
DMF at dose levels of 25 mg/kg (sub optimal) and 50 mg/kg (optimal) are administered by the oral route, once daily (QD).
Laquinimod at dose levels of 0.12 and 0.06 mg/kg are administered by the oral route, once daily (QD).
Both DMF and laquinimod are administered prophylactic from disease induction—Day 1 until termination of the study.

Induction of EAE:

EAE is induced by subcutaneous injection of encephalitogenic emulsion at a volume of 0.2 ml/mouse in the right flank. On the day of induction, pertussis toxin is injected i.p. at a volume dose of 0.2 ml/mouse. The injection of the pertussis toxin is repeated after 48 hours.

Test Procedure:

Day 0: Subcutaneous injection of MOG into right flank, ip injection of Pertussis toxin, beginning of daily laquinimod treatment.

Day 2: ip injection of Pertussis toxin.

Day 10: initiation of scoring of mice for EAE clinical signs.

Day 30: termination of study.

Materials:

1. DMF

2. Laquinimod

3. Mycobacterium tuberculosis (MT), Difco

4. Pertussis toxin, Sigma

5. MOG 35-55, Manufactured: Novatide

6. Complete Freund's Adjuvant (CFA), Sigma

7. Saline, Manufactured: DEMO S.A

8. Sterile double distilled water (DDW)

Experimental Animals:

Healthy, nulliparous, non-pregnant female mice of the C57BL/6 strain obtained from Harlan Animal Breeding Center, Israel are used in the study.

The animals weighed 18-22 gr, and are approximately 8 weeks old on receipt.

The body weights of the animals are recorded on the day of delivery.

Overtly healthy animals are assigned to study groups arbitrarily before treatment commenced.

The mice are individually identified by using ear tags. A color-coded card on each cage gives information including cage number, group number and identification.

EAE Induction:

EAE is induced by injecting the encephalitogenic mixture (emulsion) consisting of MOG (150.0 μg/mouse) and CFA containing M. tuberculosis (2 mg MT/mL CFA).

A volume of 0.2 ml of emulsion is injected subcutaneously into the flanks of the mice.

Pertussis toxin in 0.2 ml dosage volume is injected intraperitoneally on the day of induction and 48 hours later (total amount will be 0.1+0.1=0.2 μg/mouse).

Study Design:

The mice are allocated randomly into groups according to Table 1 below.

TABLE 1

| | Treatment | | | |
|---|---|---|---|---|
| | Groups | | | |
| | (treatment | | Administration | |
| Group | initiation) | dose/day | Route | Regimen |
| 1 | Vehicle | 10 ml/kg | Oral, QD | Both |
| 2 | Laquinimod | 0.06 mg/kg | Oral, QD | DMF and |
| 2 | Laquinimod | 0.12 mg/kg | Oral, QD | Laquinimod |
| 4 | DMF | 50 mg/kg | Oral, QD | from |
| 5 | DMF | 25 mg/kg | Oral, QD | Day 1 |
| 6 | Laquinimod + | 0.06 mg/kg + | Oral (QD) + | to 30 daily |
| | DMF | 25 mg/kg | Oral (QD) | |
| 7 | Laquinimod + | 0.06 mg/kg + | Oral (QD) + | |
| | DMF | 50 mg/kg | Oral (QD) | |

Preparation and Administration of Encephalitogenic Emulsion:

Oil Portion:

20 mg MT is added to 20 ml CFA to yield 1+1=2 mg/ml MT).

Liquid Portion:

15 mg MOG or equivalent is diluted in 10 ml Normal Saline to yield 1.5 mg/ml MOG stock solution.

The emulsion is made from equal parts of oil and liquid portions (1:1) in two syringes connected to each other with Leur lock to yield 0.75 mg/ml and 1 mg/ml MT. The emulsion is transferred to insulin syringe and 0.2 ml is injected to the right flank of each mouse. Dose=0.15 mg MOG and 0.2 mg MT/mouse.

Preparation and Administration of Pertussis Toxin:

50 μL Pertussis toxin (200 μg/ml) is added to 19.95 ml saline to yield 500 ng/ml. The pertussis toxin is administered intraperitoneally on the day of encephalitogen injection and 48 hours later (100.0 ng/0.2 ml/mouse). Total 200 ng/mouse.

Preparation and Administration of Test Articles

DMF Formulations: 0.08% Methocel/H₂O

A concentration of 2.5 and 5 mg/ml for dose levels of 25 and 50 mg/kg respectively. The mice are administered with the two concentrations of DMF (2.5 and 5 mg/ml) a volume dose level of 200 μl/mouse by the oral route for dose levels of 25 and 50 mg/kg respectively.

Laquinimod Formulations:

A concentration of 0.006 and 0.012 mg/ml laquinimod is prepared in DDW. The test formulations are stored at 2 to 8° C. until use in amber colored bottles.

The mice are administered with the two concentrations of laquinimod (0.006 and 0.012 mg/ml) a volume dose level of 200 μl/mouse by the oral route for dose levels of 0.06 and 0.12 mg/kg respectively. Both the DMF and the laquinimod formulations are administered from Day 1, once daily (QD). Six hours interval is maintained daily between administration of laquinimod and DMF.

EAE Clinical Signs:

The mice are observed daily from the 10th day post-EAE induction (first injection of MOG) and the EAE clinical signs are scored according to the grades described in Table 2 presented below.

TABLE 2
Evaluation of the EAE clinical signs

| Score | Signs | Description |
|---|---|---|
| 0 | Normal | No neurological signs. |
| | behavior | |
| 1 | Limp tail | Part or the whole tail is limp and droopy. |
| 2 | righting reflex | Animal has difficulties rolling onto his feet when |
| | | laid on its back |
| 3 | Hind leg | wobbly walk - when the mouse walks the hind |
| | weakness | legs are unsteady |
| 4 | Hind leg | The mouse drags its hind legs but is able to move |
| | paralysis | around using its fore legs |
| 5 | Full paralysis | The mouse can't move around, it looks thinner |
| | | and emaciated. |
| 6 | Moribund/ | |
| | Death | |

All mice with score 1 and above are considered sick. When the first clinical sign appears all mice are given food soaked in water, which is spread on different places on the bedding of the cages.

Interpretation of Results

Calculation of the Incidence of Disease (Disease Ratio)
The number of sick animals in each group is summed.
The incidence of disease is calculated as

$$INCIDENCE of DISEASE = ( \frac{No . of sick mice in treated group}{No . of sick mice in control group} )$$

The percent inhibition according to incidence is calculated as

$$INHIBITION ( \% ) of INCIDENCE = ( 1 - \frac{Number of sick mice in treated group}{Number of si ck mice in control group} ) \times 100$$

Calculation of the Mortality/Moribundity Rate (Mortality Ratio)
The number of dead or moribund animals in each group is summed.
The mortality of disease is calculated as

$$MORTALITY of DISEASE = ( \frac{No . of dead or moribound mice in treated group}{No . of dead or moribound mice in control group} )$$

The percent inhibition according to mortality is calculated as

$$INHIBITION ( \% ) of MORTALITY = ( 1 - \frac{Number of dead or moribound mice in treated group}{Number of dead or moribound mice in control group} ) \times 100$$

Calculation of Duration of Disease
The mean duration of disease expressed in days is calculated as

$$Mean Duration = ( \frac{\sum Duration of disease of each mouse}{No . of mice in the group} )$$

Calculation of Mean Delay in Onset of Disease
The mean onset of disease expressed in days is calculated as

$$Mean Onset = ( \frac{\sum Onset of disease of each mouse}{No . of mice in the group} )$$

The mean delay in onset of disease expressed in days is calculated by subtracting the mean onset of disease in control group from test group.

Calculation of the Mean Maximal Score and Percent Inhibition
The mean maximal score (MMS) of each group is calculated as

$$MMS = ( \frac{\sum Maximal Score of each mouse}{No . of mice in the group} )$$

The percent inhibition according to MMS is calculated as

$$INHIBITION ( \% ) of MMS = ( 1 - \frac{MMS of treated group}{No . of mice in the group} ) \times 100$$

Calculation of the Group Mean Score and Percent Inhibition
The daily scores of each mouse in the test group are summed and the individual mean daily score (IMS) is calculated as

$$IMS = ( \frac{\sum Daily score of mouse}{Observation period ( days )} )$$

The mean group score (GMS) is calculated as

$$GMS = ( \frac{Σ IMS of each mouse}{No . of mice in the group} )$$

The percent inhibition is calculated as

$$INHIBITION ( \% ) of GMS = ( 1 - \frac{GMS of treated group}{GMS of control group} ) \times 100$$

Results & Conclusions

In groups of mice, a total blocking of EAE in the group treated with DMF at optimal dose level of 50 mg/kg in combination with 0.06 mg/kg dose of laquinimod exhibits therapeutic activity at least as effective as the optimal dose of DMF (50 mg/kg) alone and 0.12 mg/kg dose of laquinimod alone according to GMS when compared to the vehicle administered control group.

In groups of mice, a total blocking of EAE in the group treated with DMF at optimal dose level of 50 mg/kg in combination with 0.06 mg/kg dose of laquinimod exhibits therapeutic activity superior to the optimal dose of DMF (50 mg/kg) alone and 0.12 mg/kg dose of laquinimod alone according to GMS when compared to the vehicle administered control group.

In groups of mice, a total blocking of EAE in the group treated with DMF at suboptimal dose level of 25 mg/kg in combination with 0.06 mg/kg dose of laquinimod exhibits activity at least as effective as the optimal dose of DMF (50 mg/kg) alone and 0.12 mg/kg dose of laquinimod alone according to GMS when compared to the vehicle administered control group.

In groups of mice, a total blocking of EAE in the group treated with DMF at suboptimal dose level of 25 mg/kg in combination with 0.06 mg/kg dose of laquinimod exhibits activity superior to the optimal dose of DMF (50 mg/kg) alone and 0.12 mg/kg dose of laquinimod alone according to GMS when compared to the vehicle administered control group.

In this study, each compound alone shows a dose dependent inhibition of disease severity. However, while the lower dosages tested (0.06 mg/kg laquinimod and 25 m/kg DMF) are moderately effective individually; the combination of DMF and laquinimod when each is administered at the respective lower dosage is so potent that it completely abrogated disease. This unexpected result suggests that lower dosages of laquinimod and DMF can be used in combination to achieve a greater than additive therapeutic result, and provides evidence that such a combination can be used for therapeutic treatment of human MS and CIS patients.

### Example 1B

### Assessment of Efficacy of Laquinimod in Combination with DMF in MOG-Induced EAE

The objective of this study was to assess the effect of combining laquinimod and DMF treatments in MOG induced EAE. The C57BL/6 strain of mouse was selected, as it is an established chronic EAE model to test for the efficacy of candidate molecules for the treatment of MS.

Materials and Methods

Disease was induced in all mice by the injection of the encephalitogenic emulsion (MOG/CFA). The test articles and vehicle were dosed daily via gavage from Day 1 until Day 30 (termination of study).

Materials:

Materials included dimethyl fumarate (Sigma), laquinimod, Pertusis toxin (Sigma, Code #2980), Myelin Oligodendrocyte Lipoprotein (Novatide, MOG-35-55), Complete Freund's Adjuvant (CFA) (Sigma, Code F5881), Mycobacterium tuberculosis H37RA MT, (Difco, Code 231141), and Methocel (methylcellulose (MC)) (Sigma, M7140-500G).

Healthy, nulliparous, non-pregnant female mice of the C57BL/6 Strain were used. The animals weighed 17-20 g on arrival, and were approximately 11 weeks of age at the time of induction. The body weights of the animals were recorded on the day of delivery. Overtly healthy animals were assigned to study groups arbitrarily before treatment commenced.

The mice were individually identified by markings on the body. Information including cage number, group number and identification were provided in a color-coded card on each cage. The test formulations were prepared by one researcher and the treatment and scoring procedure is carried out by a different researcher blind to the identification of the treatment groups.

EAE Induction:

Active EAE was induced on Day 1 via subcutaneous injection in the flanks at two injection sites. The encephalitogenic mixture (emulsion) consisting of MOG and commercial CFA containing 2 mg/mL Mycobacterium tuberculosis (MT) at a volume of 0.2 mL/mouse was injected in the right flank of the animals. Pertussis toxin was injected intraperitoneally on the day of induction and 48 hours later at dose level of 100 ng/0.2 ml/mouse. The dose of the MOG and MT was 150 μg/mouse and 200 μg/mouse respectively.

Preparation and Administration of Encephalitogenic Emulsion:

Oil Portion:

CFA (containing 1 mg/ml MT) enriched with mycobacterium tuberculosis to yield 2 mg/ml MT.

Liquid Portion:

38 mg MOG or equivalent was dissolved in 25.33 ml Normal saline to yield 1.5 mg/ml MOG.

Emulsion:

The emulsions was made from equal parts of oil (CFA containing 2.0 mg/ml MT) and liquid portions (1.5 mg MOG) in two syringes connected to each other with Leur lock to yield 0.75 mg/ml MOG. The emulsion was administered to mice of the respective groups once on Day 1 via subcutaneously injection at two injection sites (in the flanks of the mice). The dose of the MOG in all the groups was 0.15 mg/0.2 ml/mouse. The dose of the MT in all the groups was 0.2 mg/0.2 ml/mouse.

Preparation and Administration of Pertussis Toxin:

55.0 μl Pertussis toxin (200 μg/ml) or equivalent was added to 21.945 ml saline to yield 0.5 μg/ml. 0.2 ml of 0.5 μg/ml

Pertussis toxin solution was injected intraperitoneally immediately after the MOG emulsion injection for a dose level of 100 ng/mouse. Injection of the pertussis toxin was repeated in a similar manner after 48 hours.

Group Assignment:

On Day 1 the MOG EAE induced mice were allocated to the following treatment groups (15 mice/group):

TABLE 3
Group Assignment for Experiment 1B

| | Treatment | | Administration | Admin. |
|---|---|---|---|---|
| Group | groups | Dose/day | Route | Period |
| 1 | Vehicle (0.08% | 0.2 ml/mouse | Gavage bid(AM/PM) | From Day |
| | MC) | | | 1 to 30 |
| 2 | Laquinimod | 5 mg/kg/day | Gavage qd (AM) | From Day |
| | 0.08% MC | 0.2 ml/mouse | Gavage qd (PM) | 1 to 30 |
| 3 | Laquinimod | 10 mg/kg/day | Gavage qd (AM) | From Day |
| | 0.08% MC | 0.2 ml/mouse | Gavage qd (PM) | 1 to 30 |
| 4 | Laquinimod | 25 mg/kg/day | Gavage qd (AM) | From Day |
| | 0.08% MC | 0.2 ml/mouse | Gavage qd (PM) | 1 to 30 |
| 5 | DMF | 45 mg/kg => 90 mg/kg/day | Gavage bid (AM/PM) | From Day |
| | | | | 1 to 30 |
| 6* | DMF | 45 mg/kg => 90 mg/kg/day | Gavage bid(AM/PM) | From Day |
| | Laquinimod | 5 mg/kg/day | Gavage qd (AM) | 1 to 30 |
| 7* | DMF | 45 mg/kg => 90 mg/kg/day | Gavage bid(AM/PM) | From Day |
| | Laquinimod | 10 mg/kg/day | Gavage qd (AM) | 1 to 30 |

*DMF was suspended in laquinimod solution in the morning treatment
**AM/PM indicates morning/afternoon.

Test Formulations:

Laquinimod:

Laquinimod was diluted in 0.08% Methocel/H₂O. For dose level of 25.0 mg/kg laquinimod, 2.5 mg/ml stock solution was prepared (group 4). For dose level of 10.0 mg/kg laquinimod, 1.0 mg/ml stock solution was prepared (groups 3 and 7). For dose level of 5.0 mg/kg laquinimod, 0.5 mg/ml stock solution was prepared (groups 2 and 6). Laquinimod was administered to the respective groups daily, by oral gavage at a volume of 0.2 ml/mouse. Laquinimod was administered from the initiation of the study, daily to mice of groups 2, 3, 4, 6 and 7. The test formulations were stored at 2 to 8° C. until use in amber colored bottles.

DMF:

Formulation for group 5 was diluted in 0.08% Methocel/H₂O to yield a concentration of 4.5 mg/ml for dose level of 45 mg/kg. The mice were administered with DMF at volume dose level of 200 μl/mouse by the oral gavage route twice a day for a total dose level of 90 mg/kg/day.

DMF and Laquinimod Combined:

For the morning (AM) gavage (groups and 7), 4.5 mg of DMF were suspended for every 1 ml of laquinimod solution. (From the stock solutions made of laquinimod 1.0 or 0.5 mg/ml diluted in 0.08% Methocel/H₂O solutions.)

Treatments:

Mice of all the treatment groups were administered the respective test formulation from Day 1, twice daily (bid) according to experimental design.

Experimental Observations

Morbidity and Mortality:

All animals were examined once daily to detect if any are moribund. Mice were weighed once weekly.

EAE Clinical Signs:

The mice were observed daily from the 8′ day post EAE-induction and EAE clinical signs were scored. The scores were recorded on observation cards according to the grades described in Table 2 shown above.

All mice with score 1 and above were considered sick. When the first clinical sign appears all mice were given food soaked in water, which was spread on different places on the bedding of the cages. For calculation purposes, the score of animals that were sacrificed or died was carried forward.

Interpretation of Results:

Same as in Experiment 1A.

Results:

A summary of the incidence, mortality, MMS, GMS, duration of the disease, onset of the disease and the activity of each group compared to the vehicle treated control group are shown in the Summarized Table 4 below:

TABLE 4

| | | | | | | | | Mean Onset | Mean Duration |
|---|---|---|---|---|---|---|---|---|---|
| Group | Mortality | Incidence | % Inhibition 1 | MMS value | % Inhibition 2 | GMS value | % Inhibition 3 | (days) | (days) |
| 1 | 0/15 | 15/15 | — | 3.5 ± 1.0 | — | 2.1 ± 0.8 | — | 13.5 ± 1.6 | 17.0 ± 2.2 |
| 2 | 0/15 | 10/15 | 33.3 | 2.1 ± 1.7 | 40.0 | 0.8 ± 0.7 | 61.9 | 22.7 ± 6.4 | 8.0 ± 6.2 |
| | | | | | P = 0.019 | | P < 0.001 | P < 0.001 | P < 0.001 |
| 3 | 0/15 | 4/15 | 73.3 | 0.6 ± 0.2 | 82.9 | 0.2 ± 0.5 | 90.5 | 28.9 ± 3.8 | 1.8 ± 3.7 |
| | | | | | P < 0.001 | | P < 0.001 | P < 0.001 | P < 0.001 |
| 4 | 0/15 | 0/15 | 100.0 | 0.0 ± 0.0 | 100.0 | 0.0 ± 0.0 | 100.0 | 0.0 ± 0.0 | 0.0 ± 0.0 |
| | | | | | P < 0.001 | | P < 0.001 | P < 0.001 | P < 0.001 |
| 5 | 0/15 | 13/15 | 13.3 | 2.6 ± 1.4 | 25.7 | 1.4 ± 0.9 | 33.3 | 17.1 ± 6.3 | 13.4 ± 6.2 |
| | | | | | P = 0.074 | | P = 0.061 | P = 0.037 | P = 0.067 |
| 6 | 0/15 | 4/15 | 73.3 | 0.4 ± 0.7 | 88.6 | 0.1 ± 0.2 | 95.2 | 29.0 ± 4.1 | 2.0 ± 4.1 |
| | | | | | P < 0.001 | | P < 0.001 | P < 0.001 | P < 0.001 |
| 7 | 0/15 | 1/15 | 93.3 | 0.3 ± 1.0 | 93.4 | 0.1 ± 0.5 | 95.2 | 30.1 ± 3.4 | 0.9 ± 3.4 |
| | | | | | P < 0.001 | | P < 0.001 | P < 0.001 | P < 0.001 |

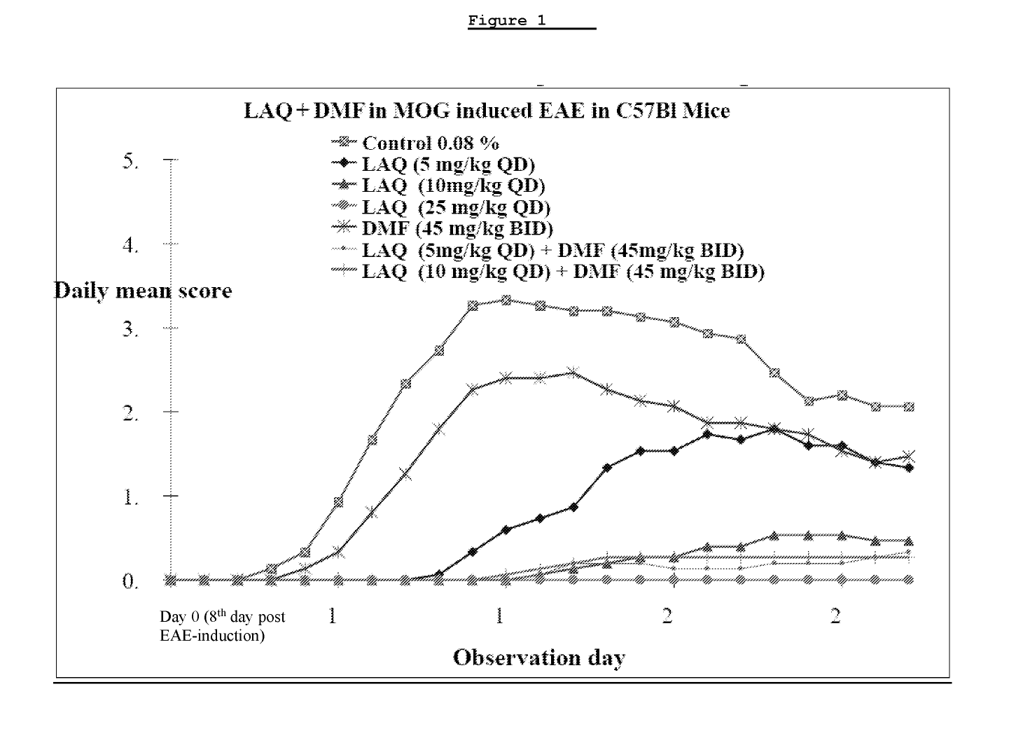

The clinical profile of the treatment groups are presented graphically in FIG. 1.

Under the conditions of the test, DMF at dose level of 45 mg/kg mouse (BID) exhibited additive activity in the suppression of EAE when tested in combination with laquinimod at dose level of 5 mg/kg. The group treated with DMF at dose level of 45 mg/kg (BID) in combination with laquinimod (5 mg/kg) exhibited 95.2% (p<0.001) activity according to GMS compared to 33.3% activity (p=0.061) in the group treated with DMF at dose level of 45 mg/kg (BID) and 61.9% activity (p<0.001) in the group treated with laquinimod at dose level of 5 mg/kg when compared to the vehicle administered control group.

The group treated with DMF at dose level of 45 mg/kg (BID) in combination with laquinimod (10 mg/kg) exhibited 95.2% activity (p<0.001) according to GMS compared to 33.3% activity (p=0.061) in the group treated with DMF at dose level of 45 mg/kg (BID) and 90.5% (p<0.001) activity in the group treated with laquinimod at dose level of 10 mg/kg when compared to the vehicle administered control group.

Laquinimod at dose level of 25 mg/kg (QD) exhibited 100% activity (p<0.001) according to GMS when compared to the vehicle administered control group.

### Example 1C

### Assessment of Efficacy of Laquinimod in Combination with DMF in MOG-Induced EAE

The objective of this study was to assess the effect of combining suboptimal dose of laquinimod with Monoethyl fumarate (MEF) or Ethyl Methyl Fumarate (EMF) in the MOG induced EAE.

Materials and Methods

Disease was induced in all mice by the injection of the encephalitogenic emulsion (MOG/CFA). The test articles and the vehicle were administrated via gavage, daily from day 1 until Day 30 (termination of study).

Materials:

Materials included Mono Ethyl Fumarate (MEF)—Dimethyl fumarate, (ACROS organics, A0277233), Ethyl Methyl Fumarate (EMF) (TA-2034), laquinimod, Pertusis toxin (Sigma, Code #2980), Myelin Oligodendrocyte Lipoprotein (Novatide, MOG-35-55), Complete Freund's Adjuvant (CFA) (Sigma, Code F5881), Mycobacterium tuberculosis H37RA MT (Difco, Code 231141), and Methocel (methylcellulose (MC) (Sigma, M7140-500G).

Healthy, nulliparous, non-pregnant female mice of the C57BL/6 Strain were used. The animals weighed 17-20 g on arrival, and were approximately 7 weeks of age at the time of induction. The body weights of the animals were recorded on the day of delivery and once weekly. Overtly healthy animals were assigned to study groups arbitrarily before treatment commenced.

EAE Induction:

Active EAE was induced on Day 1 by the subcutaneous injection in the flanks at two injection sites, the encephalitogenic mixture (emulsion) consisting of MOG and commercial CFA containing 5 mg/mL Mycobacterium tuberculosis (MT) at a volume of 0.2 mL/mouse in the right flank of the animals. The dose of the MOG and MT is 300 μg/mouse and 500 μg/mouse respectively. Pertussis toxin was injected intraperitoneally on the day of induction and 48 hours later at dose level of 150 ng/0.2 ml/mouse.

Preparation and Administration of Encephalitogenic Emulsion:

Oil Portion:

CFA (containing 5 mg/ml MT).

Liquid portion: 80 mg MOG or equivalent was dissolved in 26.67 ml Normal saline to yield 3 mg/ml MOG.

Emulsion: The emulsion was made from equal parts of oil (26.67 mL CFA containing 5.0 mg/ml MT) and liquid portions (80 mg MOG/26.67 mL PBS) in two syringes connected to each other with Leur lock. The concentration of MOG in emulsion was 1.5 mg/mL. The emulsion was transferred to insulin syringe before injection. 0.2 ml emulsion was injected into the flanks of each mouse in the study at two injection sites.

Preparation and Administration of Pertussis Toxin:

90 μL Pertussis toxin (200 μg/ml) was added to 23.91 ml PBS to yield 750 ng/ml. The pertussis toxin was administered intravenous on the day of encephalitogen injection and 48 hours later (150.0 ng/0.2 ml/mouse×2=300 ng/mouse).

Group Assignment:

The mice were allocated to the following treatment groups (15 mice/group):

TABLE 5
Group Assignment for Experiment 1C

| | Treatment | | Administration | Admin. |
|---|---|---|---|---|
| Group | groups | Dose/day | Route | Period |
| 1 | Vehicle (0.08% | | Gavage bid (AM/PM) | From Day |
| | Methyl Cellulose) | | | 1 to 30 |
| 2 | Laquinimod | 5 mg/kg/day | Gavage (AM) | From Day |
| | Vehicle (0.08% | | Gavage (PM) | 1 to 30 |
| | MC) | | | |
| 3 | Laquinimod | 25 mg/kg/day | Gavage (AM) | From Day |
| | Vehicle (0.08% | | Gavage (PM) | 1 to 30 |
| | MC) | | | |
| 4 | EMF | 135 mg/kg/day | Gavage (AM) | From Day |
| | EMF | 135 mg/kg/day | Gavage (PM) | 1 to 30 |
| 5 | MEF | 90 mg/kg | Gavage (AM) | From Day |
| | MEF | 90 mg/kg | Gavage (PM) | 1 to 30 |
| 6 | Laquinimod + | 5 mg/kg + 135 mg/kg | Gavage (AM) | From Day |
| | EMF | | | 1 to 30 |
| | EMF | 135 mg/kg | Gavage (PM) | |
| 7 | Laquinimod + | 5 mg/kg + 90 mg/kg | Gavage (AM) | From Day |
| | MEF | | | 1 to 30 |
| | MEF | 90 mg/kg | Gavage (PM) | |

Test Formulations (Preparation and Administration):

Laquinimod:

Laquinimod solutions, at concentrations of 2.5 and 0.5 mg/ml, were diluted in DDW. The test formulations were stored at 2 to 8° C. until use in amber colored bottles.

EMF and MEF:

All these formulations were diluted in 0.08% Methyl cellulose. The mice were administered twice a day with MEF at 90 mg/kg. EMF was administered at 135 mg/kg twice a day. All tested compounds were injected from day 1 till the end of experiment, at volume dose level of 200 μl/mouse.

Experimental Observations

Morbidity and Mortality:

All animals were examined once daily to detect if any are moribund.

EAE Clinical Signs:

Scoring of EAE clinical signs were initiated from the 10th day post-EAE induction and were continued daily for 30 days. The scores were recorded according to the grades described in Table 2.

All mice with score 1 and above were considered sick. Animals with score 5 for more than three days were given score 6 and sacrificed for humane reasons. For calculation purposes, the score (6) of animals that were sacrificed or died were carried forward.

Data Analysis and Calculations

Same as in Experiment 1A.

Additionally, with regards to acceptance criteria for EA induced Negative Control Group, the group should have at least 70% incidence and the MMS should be more than 2.0. Also, for calculation of mean delay in onset of disease, the onset of disease for a mouse that did not develop EAE was considered 30 days (one day after termination of study).

Results

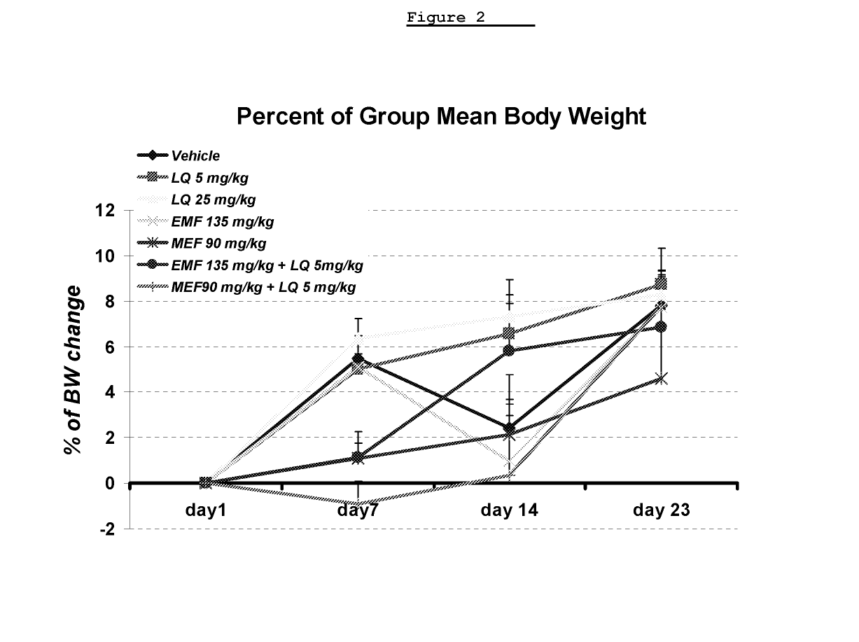

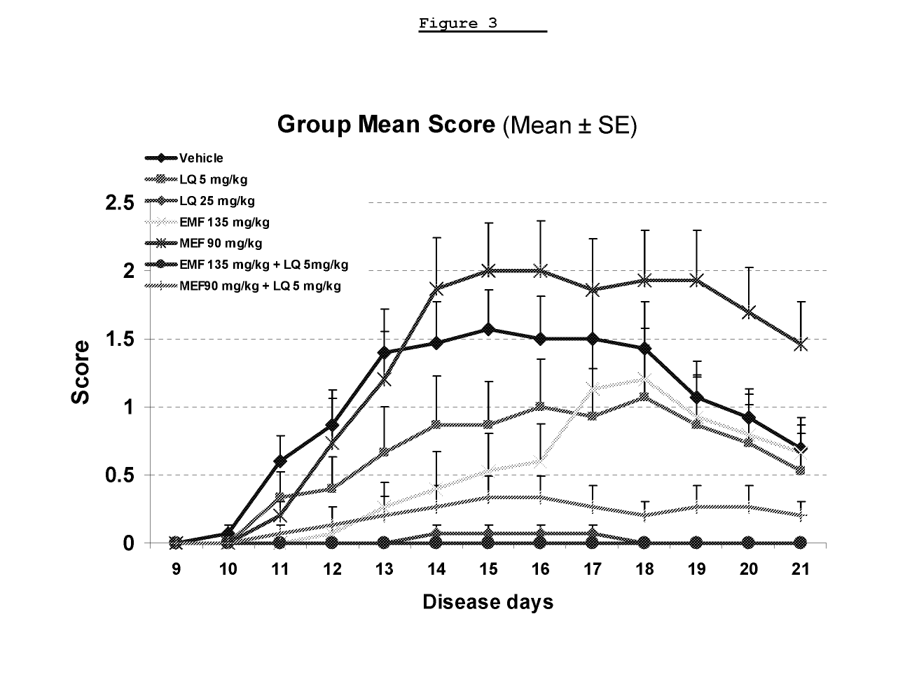

A summary of the incidence, mortality, MMS, GMS, duration of the disease, onset of the disease is shown in the summarized Table 6. The disease profile and weights of all treated groups are presented in FIG. 2 and FIG. 3. The individual daily scores of each mouse, mean maximal scores (MMS), incidence, mortality, group mean score (GMS), onset of disease, duration of disease and a figure of the clinical profile of each dosing group are presented in FIG. 3, FIG. 2, and Table 6. It was decided to interrupt this study earlier on day 21 instead day 31.

Clinical Signs and Mortality:

Since the disease was mild, no mortality was observed. Four mice were found dead in cage without clinical signs.

Incidence, Onset and Duration of Disease:

The incidence of the disease in vehicle treated group was 86.6% ( 13/15).

Laquinimod at 25 mg/kg which was used as positive control significantly reduced the incidence of disease 93.3%, as compared to vehicle treated group. Laquinimod at 5 mg/kg reduced only at 46.7% 8/15 (activity).

Treatment with EMF at 135 mg/kg BID alone, inhibit the incidence of disease at 46.6%, whereas its combination with laquinimod 5 mg/kg fully eliminated disease appearance 100% inhibition. MEF alone showed only 20% inhibition in this parameter, however in combination with LQ showed it became 66.7%.

Weight:

The differences in weight gain were correlated with the disease severity—stronger severity resulted in greater weight loss. Since the disease was mild the weight changes were not significant. The vehicle treated group showed at about 8% weigh loss on day 23. (FIG. 2)

Mean Maximal Score (MMS) and Group Mean Score (GMS):

The MMS and GMS of the vehicle treated control group were 1.9±0.3 and 0.97±0.1 respectively.

Laquinimod at 5 mg/kg administered orally daily from Day 1 exhibited only 31.6 and 34.0% suppression of EAE according to MMS and GMS respectively, compared to the vehicle treated control group. In contrast, laquinimod at 25 mg/kg showed significantly activity 94.7 and 97.9% (p≦0.001) of inhibition in MMS and GMS respectively as compared to vehicle treated group and (p≦0.05) vs LQ at 5 mg/kg.

MEF at 90 mg/kg alone was worse than Vehicle −21.1 and 26.8% of inhibition in MMS and GMS. Its combination with laquinimod at 5 mg/kg was noticeably better than MEF administration alone and showed improvement 73.7 and 79.4 of inhibition in MMS and GMS respectively as compared to vehicle treated group but not significantly.

In contrast, EMF, demonstrated an improvement on abrogation of disease 36.8 and 47.7% of inhibition in MMS and GMS as compared to vehicle treated group. This activity was similar to laquinimod at 5 mg/kg treated group.

Its combination with Laquinimod at 5 mg/kg, demonstrated highly significant improvement on abrogation of disease 100% (p≦0.001) of inhibition in both MMS and GMS respectively as compared to vehicle treated group, as well as, it was statistically significant (p≦0.05) vs EFM treatment alone (see Table 6).

### Conclusions

Laquinimod at 5 and 25 mg/kg showed dose-dependent efficacy in MOG-induced EAE.
Laquinimod at 25 mg/kg significantly ameliorated EAE clinical signs in MOG-induced EAE.
Treatment with EMF at 135 mg/kg BID alone was highly effective in this MOG induced EAE study.
Treatment with Laquinimod+EMF combination significantly ameliorated EAE clinical signs.
MEF at 90 mg/kg was not effective in alleviation of EAE disease signs.
Treatment with Laquinimod+MEF combination showed a marked activity, as compared with MEF treatment alone, but in non-significant manner.
Effectiveness of the treatment with combinations of Laquinimod+EFM at 135 mg/kg BID was similar to those which was obtained with laquinimod 25 mg/kg treatment alone.

TABLE 6

| | | | Mortality | Mean | MMS | Group | GMS | Mean Disease | |
|---|---|---|---|---|---|---|---|---|---|
| | Incidence | | Total | Maximal | Inhibition | Mean | Inhibition | Duration, | Mean Onset |
| Group | Total | % inhibition | % | Score | % | Score | % | days | days |
| Vechicle | 13/15 | 13.3 | 0 | 1.9 ± 0.3 | | 0.97 ± 0.1 | | 7.1 ± 1.1 | 12.9 ± 0.8 |
| LQ 5 mg/kg | 8/15 | 46.7 | 0 | 1.3 ± 0.4 | 31.6 | 0.64 ± 0.2 | 34.0 | 4.0 ± 1.2 | 17.3 ± 1.24 |
| LQ 25 mg/kg | 1/15 | 93.3 | 0 | 0.1 ± 0.06 | 94.7 | 0.02*** ± 0.0 | 97.9 | 0.3 ± 0.3 | 21.5 ± 0.5 |
| EFM 135 mg/kg | 8/15 | 46.7 | 0 | 1.2 ± 0.38 | 36.8 | 0.51 ± 0.2 | 47.4 | 3.3 ± 0.9 | 18.4 ± 1.0 |
| Mono EF 90 mg/kg | 12/15 | 20 | 0 | 2.3 ± 0.38 | −21.1 | 1.23 ± 0.2 | −26.8 | 7.1 ± 1.0 | 14.7 ± 1.0 |
| LQ 5 mg/kg + | 0/15 | 100 | 0 | 0 ± 0.0 | 100.0 | 0.0*** ± 0.0 | 100.0 | 0.0 ± 0.0 | 22.0 ± 0.0 |
| EFM 135 mg/kg | | | | | | | | | |
| LQ 5 mg/kg + | 5/15 | 66.7 | 0 | 0.5 ± 0.19 | 73.7 | 0.2 ± 0.1 | 79.4 | 2 ± 0.92 | 19.3 ± 1.05 |
| MonoEF 90 mg/kg | | | | | | | | | |

### Example 2A

### Assessment of Daily Administration of Laquinimod (as an Add-on Therapy to a Human Patient Already Receiving MMF, DMF, EMF or MEF

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) provides improved efficacy (provides at least the same effect with fewer adverse side effects, or an additive or more than an additive effect without unduly increasing adverse side effects or affecting the safety of the treatment) in relapsing multiple sclerosis (RMS) subjects compared to administration of the same level of MMF, DMF, EMF or MEF alone.

### Example 2B

### Assessment of Daily Administration of MMF, DMF, EMF or MEF as an Add-on Therapy to a Human Patient Already Receiving Laquinimod

Daily administration of MMF, DMF, EMF or MEF (120, 240, 360, 480, or 720 mg/day) as an add-on therapy for a human patient already receiving a suboptimal dosage of laquinimod (0.3 mg/day) provides improved efficacy (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) provides improved efficacy (provides at least the same effect with fewer adverse side effects, or an additive or more than an additive effect without unduly increasing adverse side effects or affecting the safety of the treatment) in relapsing multiple sclerosis (RMS) subjects compared to administration of the amount of laquinimod (0.3, 0.6 or 1.2 mg/day) alone.

### Example 3A

### Assessment of Efficacy of Laquinimod as an Add-on Therapy to a Human Patient Already Receiving MMF, DMF, EMF or MEF to Reduce Brain Atrophy

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) provides improved efficacy in reducing brain atrophy (provides at least the same effect with fewer adverse side effects, or an additive or more than an additive effect without unduly increasing adverse side effects or affecting the safety of the treatment) in relapsing multiple sclerosis (RMS) subjects compared to administration of the same level of MMF, DMF, EMF or MEF alone.

### Example 3B

### Assessment of Efficacy of MMF, DMF, EMF or MEF as an Add-on Therapy to a Human Patient Already Receiving Laquinimod to Reduce Brain Atrophy

Daily administration of MMF, DMF, EMF or MEF (120, 240, 360, 480, or 720 mg/day) as an add-on therapy for a human patient already receiving a suboptimal dosage of laquinimod (0.3 mg/day) reduces the amount of brain atrophy over 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) reduces the amount of brain atrophy over 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the amount of laquinimod (0.3, 0.6 or 1.2 mg/day) alone.

### Example 4A

### Assessment of Efficacy of Laquinimod as an Add-on Therapy to a Human Patient Already Receiving MMF, DMF, EMF or MEF to Reduce the Rate of Development of Clinically Definite MS and Preventing Irreversible Brain Damage

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) provides a clinically meaningful advantage and is more effective (provides an additive or more than an additive effect without unduly increasing adverse side effects or affecting the safety of the treatment) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons compared to administration of the same level of MMF, DMF, EMF or MEF alone.

### Example 4B

### Assessment of Efficacy of MMF, DMF, EMF or MEF as an Add-on Therapy to a Human Patient Already Receiving Laquinimod to Reduce the Rate of Development of Clinically Definite MS and Preventing Irreversible Brain Damage

Daily administration of MMF, DMF, EMF or MEF (120, 240, 360, 480, or 720 mg/day) as an add-on therapy for a human patient already receiving a suboptimal dosage of laquinimod (0.3 mg/day) provides a clinically meaningful advantage and is more effective (provides an additive or more than an additive effect without unduly increasing adverse side effects or affecting the safety of the treatment) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons compared to administration of an higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) provides a clinically meaningful advantage and is more effective (provides an additive or more than an additive effect without unduly increasing adverse side effects or affecting the safety of the treatment) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons compared to administration of the amount of laquinimod (0.3, 0.6 or 1.2 mg/day) alone.

### Example 5A

### Assessment of Efficacy of Laquinimod as an Add-on Therapy to a Human Patient Already Receiving MMF, DMF, EMF or MEF to Reduce Cumulative Number of New T1 Gd-Enhancing Lesions

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) reduces the cumulative number of new T1 Gd-enhancing lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of MMF, DMF, EMF or MEF alone.

### Example 5B

### Assessment of Efficacy of MMF, DMF, EMF or MEF as an Add-on Therapy to a Human Patient Already Receiving Laquinimod to Reduce Cumulative Number of New T1 Gd-Enhancing Lesions

Daily administration of MMF, DMF, EMF or MEF (120, 240, 360, 480, or 720 mg/day) as an add-on therapy for a human patient already receiving a suboptimal dosage of laquinimod (0.3 mg/day) reduces the cumulative number of new T1 Gd-enhancing lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) reduces the cumulative number of new T1 Gd-enhancing lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the amount of laquinimod (0.3, 0.6 or 1.2 mg/day) alone.

### Example 6A

### Assessment of Efficacy of Laquinimod as an Add-on Therapy to a Human Patient Already Receiving MMF, DMF, EMF or MEF to Reduce Cumulative Number of New T2 Lesions

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) reduces the cumulative number of new T2 lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of MMF, DMF, EMF or MEF alone.

### Example 6B

### Assessment of Efficacy of MMF, DMF, EMF or MEF as an Add-on Therapy to a Human Patient Already Receiving Laquinimod to Reduce Cumulative Number of New T2 Lesions

Daily administration of MMF, DMF, EMF or MEF (120, 240, 360, 480, or 720 mg/day) as an add-on therapy for a human patient already receiving a suboptimal dosage of laquinimod (0.3 mg/day) reduces the cumulative number of new T2 lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) reduces the cumulative number of new T2 lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the amount of laquinimod (0.3, 0.6 or 1.2 mg/day) alone.

### Example 7A

### Assessment of Efficacy of Laquinimod as an Add-on Therapy to a Human Patient Already Receiving MMF, DMF, EMF or MEF to Reduce Cumulative Number of New T1 Hypointense Lesions

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) reduces the cumulative number of new T1 hypointense lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of MMF, DMF, EMF or MEF alone.

### Example 7B

### Assessment of Efficacy of MMF, DMF, EMF or MEF as an Add-on Therapy to a Human Patient Already Receiving Laquinimod to Reduce Cumulative Number of New T1 Hypointense Lesions

Daily administration of MMF, DMF, EMF or MEF (120, 240, 360, 480, or 720 mg/day) as an add-on therapy for a human patient already receiving a suboptimal dosage of laquinimod (0.3 mg/day) reduces the cumulative number of new T1 hypointense lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) reduces the cumulative number of new T1 hypointense lesions as measured at 2, 4 and 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the amount of laquinimod (0.3, 0.6 or 1.2 mg/day) alone.

### Example 8A

### Assessment of Efficacy of Laquinimod as an Add-on Therapy to a Human Patient Already Receiving MMF, DMF, EMF or MEF to Reduce Total Volume of T1 Gd-Enhancing Lesions

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) reduces the total volume of T1 Gd-enhancing lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of MMF, DMF, EMF or MEF alone.

### Example 8B

### Assessment of Efficacy of MMF, DMF, EMF or MEF as an Add-on Therapy to a Human Patient Already Receiving Laquinimod to Reduce Total Volume of T1 Gd-Enhancing Lesions

Daily administration of MMF, DMF, EMF or MEF (120, 240, 360, 480, or 720 mg/day) as an add-on therapy for a human patient already receiving a suboptimal dosage of laquinimod (0.3 mg/day) reduces the total volume of T1 Gd-enhancing lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg/day) of laquinimod alone.

In addition daily administration of MMF, DMF, EMF or MEF (120, 240, 360, 480, or 720 mg/day) as an add-on therapy for a human patient already receiving an amount of laquinimod (0.3, 0.6 or 1.2 mg/day) reduces the total volume of T1 Gd-enhancing lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the amount of laquinimod (0.3, 0.6 or 1.2 mg/day) alone.

### Example 9A

### Assessment of Efficacy of Laquinimod as an Add-on Therapy to a Human Patient Already Receiving MMF, DMF, EMF or MEF to Reduce Total Volume of T2 Lesions

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) reduces the total volume of T2 lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the same level of MMF, DMF, EMF or MEF alone.

### Example 9B

### Assessment of Efficacy of MMF, DMF, EMF or MEF as an Add-on Therapy to a Human Patient Already Receiving Laquinimod to Reduce Total Volume of T2 Lesions

Daily administration of MMF, DMF, EMF or MEF (120, 240, 360, 480, or 720 mg/day) as an add-on therapy for a human patient already receiving a suboptimal dosage of laquinimod (0.3 mg/day) reduces the total volume of T2 lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) reduces the total volume of T2 lesions as measured at 6 months (provides at least the same effect with fewer adverse side effects, or an additive or more than an additive effect without unduly increasing adverse side effects or affecting the safety of the treatment) in relapsing multiple sclerosis (RMS) subjects compared to administration of the amount of laquinimod (0.3, 0.6 or 1.2 mg/day) alone.

### Example 10A

### Assessment of Efficacy of Laquinimod as an Add-on Therapy to a Human Patient Already Receiving MMF, DMF, EMF or MEF to Reduce Annualized Relapse Rate

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) reduces annualized relapse rate (provides at least the same effect with fewer adverse side effects, or an additive or more than an additive effect without unduly increasing adverse side effects or affecting the safety of the treatment) in relapsing multiple sclerosis (RMS) subjects compared to administration of the same level of MMF, DMF, EMF or MEF alone.

### Example 10B

### Assessment of Efficacy of MMF, DMF, EMF or MEF as an Add-on Therapy to a Human Patient Already Receiving Laquinimod to Reduce Annualized Relapse Rate

Daily administration of MMF, DMF, EMF or MEF (120, 240, 360, 480, or 720 mg/day) as an add-on therapy for a human patient already receiving a suboptimal dosage of laquinimod (0.3 mg/day) reduces annualized relapse rate (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) reduces annualized relapse rate (provides at least the same effect with fewer adverse side effects, or an additive or more than an additive effect without unduly increasing adverse side effects or affecting the safety of the treatment) in relapsing multiple sclerosis (RMS) subjects compared to administration of the amount of laquinimod (0.3, 0.6 or 1.2 mg/day) alone.

### Example 11A

### Assessment of Efficacy of Laquinimod as an Add-on Therapy to a Human Patient Already Receiving MMF, DMF, EMF or MEF to Reduce Accumulation of Physical Disability

Daily administration of laquinimod (p.o., 0.3 mg/day, 0.6 mg/day or 1.2 mg/day) as an add-on therapy for a human patient already receiving MMF, DMF, EMF or MEF (120, 240, 360, 480, or 720 mg/day) reduces accumulation of physical disability (provides at least the same effect with fewer adverse side effects, or an additive or more than an additive effect without unduly increasing adverse side effects or affecting the safety of the treatment) in relapsing multiple sclerosis (RMS) subjects compared to administration of the same level of MMF, DMF, EMF or MEF alone.

### Example 11B

### Assessment of Efficacy of MMF, DMF, EMF or MEF as an Add-on Therapy to a Human Patient Already Receiving Laquinimod to Reduce Accumulation of Physical Disability

Daily administration of MMF, DMF, EMF or MEF (120, 240, 360, 480, or 720 mg/day) as an add-on therapy for a human patient already receiving a suboptimal dosage of laquinimod (0.3 mg/day) reduces accumulation of physical disability (provides at least the same effect with fewer adverse side effects, or an additive or more than an additive effect without unduly increasing adverse side effects or affecting the safety of the treatment) in relapsing multiple sclerosis (RMS) subjects compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) reduces accumulation of physical disability (provides at least the same effect with fewer adverse side effects, or an additive or more than an additive effect without unduly increasing adverse side effects or affecting the safety of the treatment) in relapsing multiple sclerosis (RMS) subjects compared to administration of the amount of laquinimod (0.3, 0.6 or 1.2 mg/day) alone.

### Example 12A

### Assessment of Efficacy of Laquinimod as an Add-on Therapy to a Human Patient Already Receiving MMF, DMF, EMF or MEF to Delay the Conversion to Clinically Definite MS

Daily administration of laquinimod (p.o., 0.3 mg/day or 0.6 mg/day or 1.2 mg/day) as an add-on therapy for a human patient already receiving MMF, DMF, EMF or MEF (120, 240, 360, 480, or 720 mg/day) provides a clinically meaningful advantage and is more effective (provides at least the same effect with fewer adverse side effects, or an additive or more than an additive effect without unduly increasing adverse side effects or affecting the safety of the treatment) in delaying the conversion to clinically definite MS in patients presenting a CIS suggestive of MS compared to administration of the same level of MMF, DMF, EMF or MEF alone.

### Example 12B

### Assessment of Efficacy of MMF, DMF, EMF or MEF as an Add-on Therapy to a Human Patient Already Receiving Laquinimod to Delay the Conversion to Clinically Definite MS

Daily administration of MMF, DMF, EMF or MEF (120, 240, 360, 480, or 720 mg/day) as an add-on therapy for a human patient already receiving a suboptimal dosage of laquinimod (0.3 mg/day) provides a clinically meaningful advantage and is more effective (provides at least the same effect with fewer adverse side effects, or an additive or more than an additive effect without unduly increasing adverse side effects or affecting the safety of the treatment) in delaying the conversion to clinically definite MS in patients presenting a CIS suggestive of MS compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) provides a clinically meaningful advantage and is more effective (provides at least the same effect with fewer adverse side effects, or an additive or more than an additive effect without unduly increasing adverse side effects or affecting the safety of the treatment) in delaying the conversion to clinically definite MS in patients presenting a CIS suggestive of MS compared to administration of the amount of laquinimod (0.3, 0.6 or 1.2 mg/day) alone.

### Example 13A

### Assessment of Efficacy of Laquinimod as an Add-on Therapy to a Human Patient Already Receiving MMF, DMF, EMF or MEF to Reduce the Number of Adverse Events

Daily administration of laquinimod (p.o., 0.3 mg/day or 0.6 mg/day or 1.2 mg/day) as an add-on therapy for a human patient already receiving MMF, DMF, EMF or MEF (120, 240, 360, 480, or 720 mg/day) reduces the number of adverse events over a period of 2, 4 or 6 months (provides at least the same effect with fewer adverse side effects, or an additive or more than an additive effect with fewer adverse side effects) compared to administration of the same level of MMF, DMF, EMF or MEF alone.

### Example 13B

### Assessment of Efficacy of MMF, DMF, EMF or MEF as an Add-on Therapy to a Human Patient Already Receiving Laquinimod to Reduce the Number of Adverse Events

Daily administration of MMF, DMF, EMF or MEF (120, 240, 360, 480, or 720 mg/day) as an add-on therapy for a human patient already receiving a suboptimal dosage of laquinimod (0.3 mg/day) reduces the number of adverse events over a period of 2, 4 or 6 months (provides at least the same effect with fewer adverse side effects, or an additive or more than an additive effect with fewer adverse side effects) in relapsing multiple sclerosis (RMS) subjects compared to administration of a higher dosage (0.6 mg/day) of laquinimod alone.

In addition, daily administration of MMF, DMF, EMF or MEF (120, 240, 360, 480, or 720 mg/day) as an add-on therapy for a human patient already receiving an amount of laquinimod (0.3, 0.6 or 1.2 mg/day) reduces the number of adverse events over a period of 2, 4 or 6 months (provides at least the same effect with fewer adverse side effects, or an additive or more than an additive effect with fewer adverse side effects) in relapsing multiple sclerosis (RMS) subjects compared to administration of the amount of laquinimod (0.3, 0.6 or 1.2 mg/day) alone.

### Example 14

### Assessment of Efficacy of Daily Administration of Laquinimod and MMF, DMF, EMF or MEF as a Combination Therapy for a Human Patient to Reduce Brain Atrophy

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient reduces the amount of brain atrophy over 6 months and provides at least the same effect with fewer adverse side effects, or an additive or more than an additive effect without unduly increasing adverse side effects or affecting the safety of the treatment compared to administration of the same level of MMF, DMF, EMF or MEF alone and/or the same level of laquinimod alone.

### Example 15

### Assessment of Efficacy of Daily Administration of Laquinimod and MMF, DMF, EMF or MEF as a Combination Therapy for a Human Patient to Reduce Cumulative Number of New T1 Gd-Enhancing Lesions

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient reduces the cumulative number of new T1 Gd-enhancing lesions as measured at 2, 4 and 6 months and provides at least the same effect with fewer adverse side effects, or an additive or more than an additive effect without unduly increasing adverse side effects or affecting the safety of the treatment compared to administration of the same level of MMF, DMF, EMF or MEF alone and/or the same level of laquinimod alone.

### Example 16

### Assessment of Efficacy of Daily Administration of Laquinimod and MMF, DMF, EMF or MEF as a Combination Therapy for a Human Patient to Reduce Cumulative Number of New T2 Lesions

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient reduces the cumulative number of new T2 lesions as measured at 2, 4 and 6 months and provides at least the same effect with fewer adverse side effects, or an additive or more than an additive effect without unduly increasing adverse side effects or affecting the safety of the treatment compared to administration of the same level of MMF, DMF, EMF or MEF alone and/or the same level of laquinimod alone.

### Example 17

### Assessment of Efficacy of Daily Administration of Laquinimod and MMF, DMF, EMF or MEF as a Combination Therapy for a Human Patient to Reduce Cumulative Number of New T1 Hypointense Lesions

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient reduces the cumulative number of new T1 hypointense lesions as measured at 2, 4 and 6 months and provides at least the same effect with fewer adverse side effects, or an additive or more than an additive effect without unduly increasing adverse side effects or affecting the safety of the treatment compared to administration of the same level of MMF, DMF, EMF or MEF alone and/or the same level of laquinimod alone.

### Example 18

### Assessment of Efficacy of Daily Administration of Laquinimod and MMF, DMF, EMF or MEF as a Combination Therapy for a Human Patient to Reduce Total Volume of T1 Gd-Enhancing Lesions

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient reduces the total volume of T1 Gd-enhancing lesions as measured at 6 months and provides at least the same effect with fewer adverse side effects, or an additive or more than an additive effect without unduly increasing adverse side effects or affecting the safety of the treatment compared to administration of the same level of MMF, DMF, EMF or MEF alone and/or the same level of laquinimod alone.

### Example 19

### Assessment of Efficacy of Daily Administration of Laquinimod and MMF, DMF, EMF or MEF as a Combination Therapy for a Human Patient to Reduce Total Volume of T2 Lesions

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient reduces the total volume of T2 lesions as measured at 6 months and provides at least the same effect with fewer adverse side effects, or an additive or more than an additive effect without unduly increasing adverse side effects or affecting the safety of the treatment compared to administration of the same level of MMF, DMF, EMF or MEF alone and/or the same level of laquinimod alone.

### Example 20

### Assessment of Efficacy of Daily Administration of Laquinimod and MMF, DMF, EMF or MEF as a Combination Therapy for a Human Patient to Reduce Annualized Relapse Rate

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient reduces annualized relapse rate and provides at least the same effect with fewer adverse side effects, or an additive or more than an additive effect without unduly increasing adverse side effects or affecting the safety of the treatment compared to administration of the same level of MMF, DMF, EMF or MEF alone and/or the same level of laquinimod alone.

### Example 21

### Assessment of Efficacy of Daily Administration of Laquinimod and MMF, DMF, EMF or MEF as a Combination Therapy for a Human Patient to Reduce Accumulation of Physical Disability

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient reduces accumulation of physical disability and provides at least the same effect with fewer adverse side effects, or an additive or more than an additive effect without unduly increasing adverse side effects or affecting the safety of the treatment compared to administration of the same level of MMF, DMF, EMF or MEF alone and/or the same level of laquinimod alone. Accumulation of physical disability measured by the time to confirmed progression of EDSS during the study period (A confirmed progression of EDSS is defined as a 1 point increase from baseline on EDSS score if baseline EDSS was between 0 and 5.0, or a 0.5 point increase if baseline EDSS was 5.5. Progression cannot be confirmed during a relapse.

### Example 22

### Assessment of Efficacy of Daily Administration of Laquinimod and MMF, DMF, EMF or MEF as a Combination Therapy for a Human Patient to Delay the Conversion to Clinically Definite MS

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient provides a clinically meaningful advantage and is more effective (provides at least the same effect with fewer adverse side effects, or an additive or more than an additive effect without unduly increasing adverse side effects or affecting the safety of the treatment) in delaying the conversion to clinically definite MS in patients presenting a CIS suggestive of MS than when MMF, DMF, EMF or MEF and/or laquinimod is administered alone (at the same dose).

### Example 23

### Assessment of Efficacy of Daily Administration of Laquinimod and MMF, DMF, EMF or MEF as a Combination Therapy for a Human Patient to Reduce the Rate of Development of Clinically Definite MS and Preventing Irreversible Brain Damage

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient provides a clinically meaningful advantage and is more effective (provides at least the same effect with fewer adverse side effects, or an additive or more than an additive effect without unduly increasing adverse side effects or affecting the safety of the treatment) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when MMF, DMF, EMF or MEF and/or laquinimod is administered alone (at the same dose).

### Example 24

### Assessment of Adverse Events from Daily Administration of Laquinimod and MMF, DMF, EMF or MEF as a Combination Therapy for a Human Patient

Daily administration of laquinimod (p.o., 0.3 mg/day or 0.6 mg/day or 1.2 mg/day) and MMF, DMF, EMF or MEF (120, 240, 360, 480, or 720 mg/day) as a combination therapy for a human patient results in a reduced number of adverse events over a period of 2, 4 or 6 months compared to the same dose of MMF, DMF, EMF or MEF and/or laquinimod.

### Example 25

### Assessment of Daily Administration of Laquinimod (0.3 mg/Day) and MMF, DMF, EMF or MEF as a Combination Therapy for Relapsing Multiple Sclerosis (RMS) Patients

Daily administration of laquinimod (p.o., 0.3 mg/day) and MMF, DMF, EMF or MEF (120, 240, 360, 480, or 720 mg/day) provides a clinically meaningful advantage and is more effective (provides an additive effect or more than an additive effect) in treating relapsing multiple sclerosis (RMS) patients than when each agent is administered alone (at the same dose) in the following manner:

Daily administration of laquinimod (p.o., 0.3 mg/day) and MMF, DMF, EMF or MEF is more effective (provides an additive effect or more than an additive effect) in reducing the number of confirmed relapses and therefore the relapse rate, in relapsing multiple sclerosis (RMS) patients compared to administration of the same level of MMF, DMF, EMF or MEF alone or laquinimod (p.o., 0.6 mg/day).

Daily administration of laquinimod (p.o., 0.3 mg/day) and MMF, DMF, EMF or MEF is also more effective (provides an additive effect or more than an additive effect) in reducing the accumulation of physical disability in relapsing multiple sclerosis (RMS) patients, as measured by the time to confirmed progression of EDSS, compared to administration of the same level of MMF, DMF, EMF or MEF alone or laquinimod (p.o., 0.6 mg/day).

Daily administration of laquinimod (p.o., 0.3 mg/day) and MMF, DMF, EMF or MEF is also more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number of new T2 lesions, change in brain volume, the cumulative number of new T1 hypointense lesions on T1-weight images (black holes), presence or absence of GdE lesions, change in total volume of T1 Gd-enhancing lesions, and/or change in total volume of T2 lesions, compared to administration of the same level of MMF, DMF, EMF or MEF alone or laquinimod (p.o., 0.6 mg/day).

Daily administration of laquinimod (p.o., 0.3 mg/day) and MMF, DMF, EMF or MEF is more effective (provides an additive effect or more than an additive effect) in reducing brain atrophy in relapsing multiple sclerosis (RMS) patients, compared to administration of the same level of MMF, DMF, EMF or MEF alone or laquinimod (p.o., 0.6 mg/day).

Daily administration of laquinimod (p.o., 0.3 mg/day) and MMF, DMF, EMF or MEF is more effective (provides an additive effect or more than an additive effect) in reducing the frequency of relapses, the frequency of clinical exacerbation, the risk for confirmed progression, and the time to confirmed disease progression in relapsing multiple sclerosis (RMS) patients, compared to administration of the same level of MMF, DMF, EMF or MEF alone or laquinimod (p.o., 0.6 mg/day).

### Example 26

### Assessment of Daily Administration of Laquinimod (0.6 mg/Day) and MMF, DMF, EMF or MEF as a Combination Therapy for Relapsing Multiple Sclerosis (RMS) Patients

Daily administration of laquinimod (p.o., 0.6 mg/day) and MMF, DMF, EMF or MEF (120, 240, 360, 480, or 720 mg/day) provides a clinically meaningful advantage and is more effective (provides an additive effect or more than an additive effect) in treating relapsing multiple sclerosis (RMS) patients than when each agent is administered alone (at the same dose) in the following manner:

Daily administration of laquinimod (p.o., 0.6 mg/day) and MMF, DMF, EMF or MEF is more effective (provides an additive effect or more than an additive effect) in reducing the number of confirmed relapses and therefore the relapse rate, in relapsing multiple sclerosis (RMS) patients compared to administration of the same level of each agent alone.

Daily administration of laquinimod (p.o., 0.6 mg/day) and MMF, DMF, EMF or MEF is also more effective (provides an additive effect or more than an additive effect) in reducing the accumulation of physical disability in relapsing multiple sclerosis (RMS) patients, as measured by the time to confirmed progression of EDSS, compared to administration of the same level of each agent alone.

Daily administration of laquinimod (p.o., 0.6 mg/day) and MMF, DMF, EMF or MEF is also more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number of new T2 lesions, change in brain volume, the cumulative number of new T1 hypointense lesions on T1-weight images (black holes), presence or absence of GdE lesions, change in total volume of T1 Gd-enhancing lesions, and/or change in total volume of T2 lesions, compared to administration of the same level of each agent alone.

Daily administration of laquinimod (p.o., 0.6 mg/day) and MMF, DMF, EMF or MEF is more effective (provides an additive effect or more than an additive effect) in reducing brain atrophy in relapsing multiple sclerosis (RMS) patients, compared to administration of the same level of each agent alone.

Daily administration of laquinimod (p.o., 0.6 mg/day) and MMF, DMF, EMF or MEF is more effective (provides an additive effect or more than an additive effect) in reducing the frequency of relapses, the frequency of clinical exacerbation, the risk for confirmed progression, and the time to confirmed disease progression in relapsing multiple sclerosis (RMS) patients, compared to administration of the same level of each agent alone.

### Example 27

### Assessment of Daily Administration of Laquinimod (1.2 mg/Day) and MMF, DMF, EMF or MEF as a Combination Therapy for Relapsing Multiple Sclerosis (RMS) Patients

Daily administration of laquinimod (p.o., 1.2 mg/day) and MMF, DMF, EMF or MEF (120, 240, 360, 480, or 720 mg/day) provides a clinically meaningful advantage and is more effective (provides an additive effect or more than an additive effect) in treating relapsing multiple sclerosis (RMS) patients than when each agent is administered alone (at the same dose) in the following manner:

Daily administration of laquinimod (p.o., 1.2 mg/day) and MMF, DMF, EMF or MEF is more effective (provides an additive effect or more than an additive effect) in reducing the number of confirmed relapses and therefore the relapse rate, in relapsing multiple sclerosis (RMS) patients compared to administration of the same level of each agent alone.

Daily administration of laquinimod (p.o., 1.2 mg/day) and MMF, DMF, EMF or MEF is also more effective (provides an additive effect or more than an additive effect) in reducing the accumulation of physical disability in relapsing multiple sclerosis (RMS) patients, as measured by the time to confirmed progression of EDSS, compared to administration of the same level of each agent alone.

Daily administration of laquinimod (p.o., 1.2 mg/day) and MMF, DMF, EMF or MEF is also more effective (provides an additive effect or more than an additive effect) in reducing MRI-monitored disease activity in relapsing multiple sclerosis (RMS) patients, as measured by the cumulative number of T1 Gd-enhancing lesions on T1-weighted images, the cumulative number of new T2 lesions, change in brain volume, the cumulative number of new T1 hypointense lesions on T1-weight images (black holes), presence or absence of GdE lesions, change in total volume of T1 Gd-enhancing lesions, and/or change in total volume of T2 lesions, compared to administration of the same level of each agent alone. Daily administration of laquinimod (p.o., 1.2 mg/day) and MMF, DMF, EMF or MEF is more effective (provides an additive effect or more than an additive effect) in reducing brain atrophy in relapsing multiple sclerosis (RMS) patients, compared to administration of the same level of each agent alone.

Daily administration of laquinimod (p.o., 1.2 mg/day) and MMF, DMF, EMF or MEF is more effective (provides an additive effect or more than an additive effect) in reducing the frequency of relapses, the frequency of clinical exacerbation, the risk for confirmed progression, and the time to confirmed disease progression in relapsing multiple sclerosis (RMS) patients, compared to administration of the same level of each agent alone.

### Example 28

### Laquinimod Combination Therapy for Multiple Sclerosis Patients

Compounds of formula (I) other than MMF, DMF, EMF or MEF in combination with an amount of laquinimod are administered to patients afflicted with a form of multiple sclerosis.

The compounds of formula (I) exhibit similar activities in combination with laquinimod as the combination of MMF, DMF, EMF or MEF with laquinimod described above.

### Example 29

### Compound of Formula (I) Synthesis

The monoalkyl and dialkyl fumarates of the present invention are prepared by methods known to those of ordinary skill in the art. Variations on the following general synthetic methods will be readily apparent to those of ordinary skill in the art and are used to prepare the compounds of the method of the present invention. The synthesis of the monoalkyl and dialkyl fumarates of the present invention are carried out according to general Scheme 1 or Scheme 2.

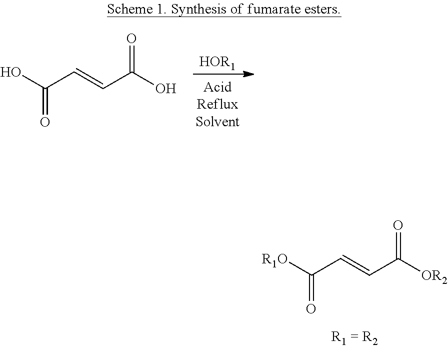

Fumuraic acid is stirred in the presence of the appropriate alcohol and catalytic acid at reflux to form the desired fumarate ester.

The solvent is a non-reactive co-solvent that does not chemically interfere with the reaction. Non-limiting examples of non-reactive co-solvents include methyl acetate, ethyl acetate, isopropyl acetate, n-butyl acetate, isobutyl acetate, acetone, methyl ethyl ketone, methyl isobutyl ketone, tetrahydrofuran, 2-methyltetrahydrofuran, acetonitrile, methyl t-butyl ether, dibutyl ether, cyclopentyl methyl ether, anisole, toluene, xylene, heptanes, and mixtures thereof. In one embodiment, the non-reactive co-solvents include methyl acetate, ethyl acetate, isopropyl acetate, n-butyl acetate, isobutyl acetate, acetone, methyl ethyl ketone, methyl isobutyl ketone, dibutyl ether, anisole, toluene, heptanes, and mixtures thereof. Alternatively, the solvent is absent and excess alcohol (HOR₁) acts as the solvent.

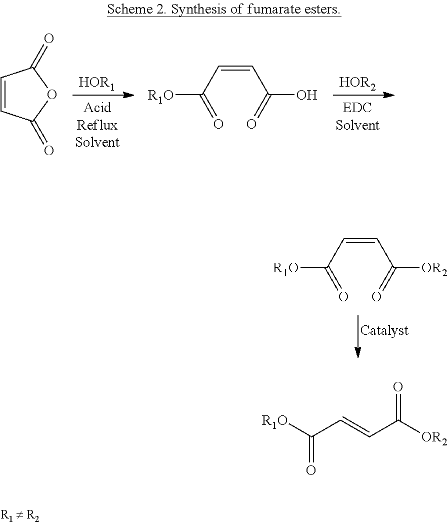

Maleic anhydride is stirred in the presence of the appropriate alcohol (1 eq.) and catalytic acid at reflux to form the desired monoalkyl maleate. The monoalkyl maleate is stirred in the presence of the appropriate alcohol and N-(3-dimethylaminopropyl)-N′-ethylcarbodiimide hydrochloride to form the desired dialkyl maleate, which isomerizes to the desired fumarate ester. Isomerization occurs in the presence of, for example, various amine catalysts, acid catalysts or bromine.

The solvent is a non-reactive co-solvent that does not chemically interfere with the reaction. Non-limiting examples of non-reactive co-solvents include methyl acetate, ethyl acetate, isopropyl acetate, n-butyl acetate, isobutyl acetate, acetone, methyl ethyl ketone, methyl isobutyl ketone, tetrahydrofuran, 2-methyltetrahydrofuran, acetonitrile, methyl t-butyl ether, dibutyl ether, cyclopentyl methyl ether, anisole, toluene, xylene, heptanes, and mixtures thereof. In one embodiment, the non-reactive co-solvents include methyl acetate, ethyl acetate, isopropyl acetate, n-butyl acetate, isobutyl acetate, acetone, methyl ethyl ketone, methyl isobutyl ketone, dibutyl ether, anisole, toluene, heptanes, and mixtures thereof.

Suitable reaction parameters have been described in WO 2012/170923 A1; Organic Syntheses, Coll. Vol. 7, p.93 (1990); Vol. 63, p.183 (1985); and J. Chem. Educ., 1991, 68 (12), p 1050, the content of each of which are hereby incorporated by reference. The compounds used in the method of the present invention may also be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Fumaric acid (Catalog # W248800, Sigma-Aldrich, St. Louis, Mo., USA), dimethyl fumarate (Catalog # D95654, Sigma-Aldrich, St. Louis, Mo., USA), monomethyl fumarate (Catalog #651419, Sigma-Aldrich, St. Louis, Mo., USA), and monoethyl fumarate (Catalog #128422, Sigma-Aldrich, St. Louis, Mo., USA) are commercially available. Maleic anhydride is also commercially available (Catalog # M625, Sigma-Aldrich, St. Louis, Mo., USA).

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

Isotopically-labeled compounds can generally be prepared by conventional techniques known to those skilled in the art using appropriate isotopically-labeled reagents in place of the non-labeled reagents employed.

### REFERENCES

1. Berdyshev et al. (2009) “FTY720 inhibits ceramide synthases and up-regulates dihydrosphingosine 1-phosphate formation in human lung endothelial cells.” Journal of Biological Chemistry, 284(9):5467-77.
2. Billich et al. (2003) “Phosphorylation of the immunomodulatory drug FTY720 by sphingosine kinases.” J Biol Chem, 278(48):47408-15.
3. Bjartmar and Fox (2002) “Pathological mechanisms and disease progression of multiple sclerosis: therapeutic implication”, Drugs of Today, 38:7-29.
4. Brex et al., (2002) “A longitudinal study of abnormalities on MRI and disability from multiple sclerosis.” N Engl J Med., Jan. 17, 2002 346(3):158-64.
5. Brod et al. (2000) Annals of Neurology, 47:127-131.
6. Brück et al., (2011) “Insight into the mechanism of laquinimod action”, J. Neurol Sci. 306:173-179.
7. De Stefano et al. (1999) “Evidence of early axonal damage in patients with multiple sclerosis.” Neurology, 52(Suppl 2):A378.
8. Dunitz, M. Multiple sclerosis therapeutics, Ed. Rudick and Goodkin. London: Taylor & Francis, 1999.
9. EMEA Guideline on Clinical Investigation of Medicinal Products for the Treatment of Multiple Sclerosis (CPMP/EWP/561/98 Rev. 1, Nove.2006).
10. Freireich et al. (1966) “Quantitative comparison to toxicity of anticancer agents in mouse, rat, hamster, dog, monkey and man.” Cancer Chemother Rep, 50:219-244.
11. Frohman et al. (2003) “The utility of MRI in suspected MS: report of the Therapeutics and Technology Assessment Subcommittee of the American Academy of Neurology”, Neurology. Sep. 9, 2003, 61(5):602-11.
12. Golder W, (2007) “Magnetic resonance spectroscopy in clinical oncology”, Onkologie. 27(3): 304-9.
13. Grossman et al., (1994) Magnetization transfer: theory and clinical applications in neuroradiology”, RadioGraphics. 14:279-290.
14. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999<http://www.fda.gov/cber/gdlns/metabol.pdf>.
15. Gurevich et al. (2010) “Laquinimod suppress antigen presentation in relapsing-remitting multiple sclerosis: invitro high-throughput gene expression study.” J Neuroimmunol. 2010 April 15; 221(1-2):87-94. Epub 2010 March 27.
16. Hauser et al. (1983) “Intensive immunosuppression in progressive multiple sclerosis,” New England Journal of Medicine, 308:173-180.
17. Hla et al. (2001) “Lysophospholipids—receptor revelations.” Science, 294(5548):1875-8.
18. Hohlfeld et al. (2000) “The neuroprotective effect of inflammation: implications for the therapy of multiple sclerosis.” J Neuroimmunol., 107:161-166.
19. Horga and Montalban 06/04/2008; Expert Rev Neurother., 2008; 8(5):699-714.
20. Kleinschmidt-DeMasters et al. (2005) New England Journal of Medicine, 353:369-379.
21. Kurtzke J F, (1983) “Rating neurologic impairment in multiple sclerosis: an expanded disability status scale (EDSS)”, Neurology 33(11):1444-1452.
22. Langer-Gould et al. (2005) New England Journal of Medicine, 353:369-379.
23. McDonald, (2001) “Guidelines from the International Panel on the Diagnosis of Multiple Sclerosis” Ann. Neurol., 50:121-127.
24. Mehta et al. (1996) “Magnetization transfer magnetic resonance imaging: a clinical review”, Topics in Magnetic Resonance Imaging 8(4):214-30.
25. National MS Society Website, retrieved Jul. 10, 2012<http://www.nationalmssociety.org/ms-clinical-care-network/researchers/clinical-study-measures/index.aspx>
26. Neuhaus et al. (2003) “Immunomodulation in multiple sclerosis: from immunosuppression to neuroprotection.” Trends Pharmacol Sci., 24:131-138.
27. Noseworthy et al. (2000) “Multiple sclerosis”, N Engl J Med., 343:938-952.
28. Paugh et al. (2003) “The immunosuppressant FTY720 is phosphorylated by sphingosine kinase type 2.” FEBS Lett, 554(1-2):189-93.
29. Paugh et al. (2006). “Sphingosine and its analog, the immunosuppressant 2-amino-2-(2-[4-octylphenyl]ethyl)-1,3-propanediol, interact with the CB1 cannabinoid receptor.” Mol Pharmacol., 70(1):41-50.
30. Payne et al. (2007) “The immunosuppressant drug FTY720 inhibits cytosolic phospholipase A2 independently of sphingosine-1-phosphate receptors.” Blood, 109(3):1077-85. doi:10.1182/blood-2006-03-011437.
31. PCT International Application Publication No. WO 2007/0047863, published Apr. 26, 2007.
32. PCT International Application Publication No. WO 2007/0146248, published Dec. 21, 2007.
33. Polman et al. (2005) “Treatment with laquinimod reduces development of active MRI lesions in relapsing MS.” Neurology. 64:987-991.
34. Polman et al. (2011) “Diagnostic Criteria for Multiple Sclerosis: 2010 Revisions to the McDonald Criteria.” Ann Neural, 69:292-302.
35. Polman et al., (2005) “Diagnostic criteria for multiple sclerosis: 2005 revisions to the McDonald Criteria.” Annals of Neurology, 58(6):840-846.
36. Poser et al. (1983) “New Diagnostic Criteria for Multiple Sclerosis: Guidelines for Research Protocols.” Annals of Neurology, March 1983, 13(3):227-230.
37. Rosen Y, (2007) “The Recent advances in magnetic resonance neurospectroscopy”, Neurotherapeutics. 27(3):330-45.
38. Rudick et al. (1999) “Use of the brain parenchymal fraction to measure whole brain atrophy in relapsing-remitting MS: Multiple Sclerosis Collaborative Research Group”. Neurology. 53:1698-1704.
39. Runstrom et al. (2002) “Laquinimod (ABR-215062) a candidate drug for treatment of Multiple Sclerosis inhibits the development of experimental autoimmune encephalomyelitis in IFN-β knock-out mice,” (Abstract), Medicon Valley Academy, Malmoe, Sweden.
40. Sanchez, et al. (2003) “Phosphorylation and action of the immunomodulator FTY720 inhibits vascular endothelial cell growth factor-induced vascular permeability.” The Journal of biological chemistry, 278(47):47281-90.
41. Sandberg-Wollheim et al. (2005) “48-week open safety study with high-dose oral laquinimod in patients.” Mult Scler. 11:S154 (Abstract).
42. The National MS Society (USA), The Disease Modifying Drug Brochure, Oct. 19, 2006.
43. U.S. Pat. No. 6,077,851, issued Jun. 20, 2000 (Bjork et al).
44. U.S. Pat. No. 7,589,208, issued Sep. 15, 2009 (Jansson et al).
45. Vollmer et al. (2008) “Glatiramer acetate after induction therapy with mitoxantrone in relapsing multiple sclerosis.” Multiple Sclerosis, 00:1-8.
46. Yang et al. (2004) “Laquinimod (ABR-215062) suppresses the development of experimental autoimmune encephalomyelitis, modulates the Th1/Th2 balance and induces the Th3 cytokine TGF-β in Lewis rats.” J. Neuroimmunol. 156:3-9.

What is claimed is:

1. A method of treating a subject afflicted with a form of multiple sclerosis (MS) or presenting a clinically isolated syndrome (CIS) comprising periodically administering to the subject
a) an amount of laquinimod or a pharmaceutically acceptable salt thereof, and
b) an amount of a compound of formula (I): 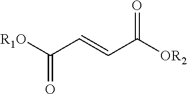
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, or
a pharmaceutically acceptable salt thereof,
wherein the amounts when taken together are more effective to treat the subject than when each agent at the same amount is administered alone.

2. The method of claim 1, wherein the MS is relapsing MS or relapsing-remitting MS.

3. The method of claim 1 or 2, wherein the amount of laquinimod and the amount of the compound of formula (I) when taken together is effective to reduce a symptom of MS in the subject, wherein the symptom is preferably a MRI-monitored MS disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

4. The method of claim 3, wherein the amount of laquinimod and the amount of the compound of formula (I) when taken together is effective to
a) decrease or inhibit reduction of brain volume,
b) increase time to confirmed disease progression,
c) decrease abnormalities observed in whole Brain MTR histogram, or
d) reduce cognitive impairment.

5. The method of claim 4, wherein brain volume is measured by percent brain volume change (PBVC), time to confirmed disease progression is increased by 20-60%, or cognitive impairment is assessed by the Symbol Digit Modalities Test (SDMT) score.

6. The method of claim 3, wherein the accumulation of physical disability is measured by Kurtzke Expanded Disability Status Scale (EDSS) score, or by the time to confirmed disease progression as measured by Kurtzke Expanded Disability Status Scale (EDSS) score.

7. The method of claim 6, wherein the subject had an EDSS score of 0-5.5, of 1.5-4.5, or of 5.5 or greater, at baseline.

8. The method of claim 6 or 7, wherein confirmed disease progression is a 1 point or a 0.5 point increase of the EDSS score.

9. The method of claim 3, wherein impaired mobility is assessed by the Timed-25 Foot Walk test, the 12-Item MS Walking Scale (MSWS-12) self-report questionnaire, the Ambulation Index (AI), the Six-Minute Walk (6MW) Test or the Lower Extremity Manual Muscle Test (LEMMT) Test.

10. The method of claim 3, wherein general health status is assessed by the EuroQoL (EQ5D) questionnaire, Subject Global Impression (SGI) or Clinician Global Impression of Change (CGIC).

11. The method of claim 3, wherein functional status is measured by the subject's Short-Form General Health survey (SF-36) Subject Reported Questionnaire score.

12. The method of claim 3, wherein quality of life is assessed by SF-36, EQ5D, Subject Global Impression (SGI) or Clinician Global Impression of Change (CGIC).

13. The method of claim 11 or 12, wherein the subject's SF-36 mental component summary score (MSC) is improved and/or the subject's SF-36 physical component summary sore (PSC) is improved.

14. The method of claim 3, wherein fatigue is assessed by the EQ5D, the subject's Modified Fatigue Impact Scale (MFIS) score or the French valid versions of the Fatigue Impact Scale (EMIF-SEP) score.

15. The method of claim 3, wherein symptom severity on work is measured by the work productivity and activities impairment General Health (WPAI-GH) questionnaire.

16. The method of any one of claims 1-15, wherein laquinimod is laquinimod sodium.

17. The method of any one of claims 1-16, wherein the compound of formula (I) is a pharmaceutically acceptable salt thereof.

18. The method of any one of claims 1-17, wherein the laquinimod and/or the compound of formula (I) is administered via oral administration, or is administered in an aerosol, an inhalable powder, an injectable, a liquid, a solid, a capsule or a tablet form.

19. The method of any one of claims 1-18, wherein the laquinimod and/or the compound of formula (I) is administered daily.

20. The method of any one of claims 1-18, wherein the laquinimod and/or the compound of formula (I) is administered more often than once daily or less often than once daily.

21. The method of any one of claims 1-20, wherein the amount laquinimod administered is: less than 0.6 mg/day, 0.03-600 mg/day, 1-40.0 mg/day, 0.1-2.5 mg/day, 0.25-2.0 mg/day, 0.5-1.2 mg/day, 0.25 mg/day, 0.3 mg/day, 0.5 mg/day, 0.6 mg/day, 1.0 mg/day, 1.2 mg/day, 1.5 mg/day, or 2.0 mg/day.

22. The method of any one of claims 1-21, wherein the amount the compound of formula (I) administered is: 12-7200 mg/day, 120 mg/day, 360 mg/day, 480 mg/day, or 720 mg/day.

23. The method of any one of claims 1-22, wherein a loading dose of laquinimod and/or the compound of formula I) of an amount different from the intended dose is administered for a period of time at the start of the periodic administration.

24. The method of claim 23, wherein the loading dose is double the amount of the intended dose, or the loading dose is half the amount of the intended dose.

25. The method of any one of claims 1-24, wherein the subject is receiving laquinimod therapy prior to initiating the compound of formula (I) therapy.

26. The method of any one of claims 1-24, wherein the subject is receiving the compound of formula (I) therapy prior to initiating laquinimod therapy.

27. The method of claim 26, where in the subject is receiving the compound of formula (I) therapy for at least 8 weeks, at least 10 weeks, at least 24 weeks, at least 28 weeks, at least 48 weeks, or at least 52 weeks prior to initiating laquinimod therapy.

28. The method of any one of claims 1-27, further comprising administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

29. The method of any one of claims 1-28, wherein the periodic administration of laquinimod and the compound of formula (I) continues for at least 3 days, for more than 30 days, for more than 42 days, for 8 weeks or more, for at least 12 weeks, for at least 24 weeks, for more than 24 weeks, or for 6 months or more.

30. The method of any one of claims 1-29, wherein each of the amount of laquinimod when taken alone, and the amount of the compound of formula (I) when taken alone is effective to treat the subject or wherein either the amount of laquinimod when taken alone, the amount of the compound of formula (I) when taken alone, or each such amount when taken alone is not effective to treat the subject.

31. The method of any one of claims 1-30, wherein the subject is a human patient.

32. A package comprising:
a) a first pharmaceutical composition comprising an amount of laquinimod or a pharmaceutically acceptable salt thereof and a pharmaceutically acceptable carrier;
b) a second pharmaceutical composition comprising an amount of a compound of formula (I): 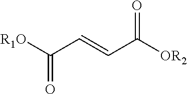
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃,
or a pharmaceutically acceptable salt thereof and a pharmaceutically acceptable carrier; and
c) instruction for use for the first and the second pharmaceutical composition together to treat a subject afflicted with a form of MS or presenting a CIS.

33. The package of claim 31, wherein the first pharmaceutical composition, the second pharmaceutical composition, or both the first and the second pharmaceutical composition are in an aerosol, an inhalable powder, an injectable, a liquid, a solid, a capsule or a tablet form.

34. The package of claim 33, wherein the tablets are coated with a coating which inhibits oxygen from contacting the core.

35. The package of claim 34, wherein the coating comprises a cellulosic polymer, a detackifier, a gloss enhancer, or pigment.

36. The package of any one of claims 32-35, wherein the first pharmaceutical composition further comprises mannitol, an alkalinizing agent, an oxidation reducing agent, a lubricant and/or a filler.

37. The package of claim 36, wherein the alkalinizing agent is meglumine.

38. The package of any one of claims 32-36, wherein the first pharmaceutical composition is stable and free of an alkalinizing agent or an oxidation reducing agent, preferably the first pharmaceutical composition is free of an alkalinizing agent and free of an oxidation reducing agent.

39. The package of any one of claims 32-38, wherein the first pharmaceutical composition is stable and free of disintegrant.

40. The package of claim 36, wherein the lubricant is present in the composition as solid particles.

41. The package of claim 36 or 40, wherein the lubricant is sodium stearyl fumarate or magnesium stearate.

42. The package of claim 36, wherein the filler is present in the composition as solid particles.

43. The package of claim 36 or 42, wherein the filler is lactose, lactose monohydrate, starch, isomalt, mannitol, sodium starch glycolate, sorbitol, lactose spray dried, lactose anhydrouse, or a combination thereof.

44. The package of any one of claims 32-43, further comprising a desiccant.

45. The package of claim 44, wherein the desiccant is silica gel.

46. The package of any one of claims 32-45, wherein the first pharmaceutical composition is stable and has a moisture content of no more than 4%.

47. The package of any one of claims 32-46, wherein laquinimod is present in the composition as solid particles.

48. The package of any one of claims 32-47, wherein the package is a sealed packaging having a moisture permeability of not more than 15 mg/day per liter.

49. The package of claim 48, wherein the sealed package is a blister pack in which the maximum moisture permeability is no more than 0.005 mg/day or a bottle.

50. The package of claim 49, wherein the bottle is closed with a heat induction liner.

51. The package of any one of claims 48-50, wherein the sealed package comprises an HDPE bottle.

52. The package of any one of claims 48-51, wherein the sealed package comprises an oxygen absorbing agent.

53. The package of claim 52, wherein the oxygen absorbing agent is iron.

54. The package of any one of claims 32-53, wherein the amount of laquinimod in the first composition is: less than 0.6 mg, 1-40.0 mg, 0.1-2.5 mg, 0.25-2.0 mg, 0.5-1.2 mg, 0.25 mg, 0.3 mg, 0.5 mg, 0.6 mg, 1.0 mg, 1.2 mg, 1.5 mg, or 2.0 mg.

55. The package of any one of claim 32-54, wherein the amount of the compound of formula (I) is: 12-7200 mg, 120 mg, 360 mg, 480 mg, or 720 mg.

56. The package of any one of claims 32-55, wherein the amount of laquinimod and the amount of the compound of formula (I) are prepared to be administered simultaneously, contemporaneously or concomitantly.

57. The package of any one of claims 32-56, for use in treating a subject afflicted with a form of MS or presenting a CIS.

58. A pharmaceutical composition comprising an amount of laquinimod or a pharmaceutically acceptable salt thereof and an amount of a compound of formula (I): 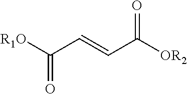
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃,
or a pharmaceutically acceptable salt thereof,
and at least one pharmaceutically acceptable carrier.

59. The pharmaceutical composition of claim 58, for use in treating a subject afflicted with MS or presenting a CIS, wherein the laquinimod and the compound of formula (I) are prepared to be administered simultaneously, contemporaneously or concomitantly.

60. The pharmaceutical composition claim 58 or 59, wherein laquinimod is laquinimod sodium.

61. The pharmaceutical composition of any one of claims 58-60, wherein the compound of formula (I) is a pharmaceutically acceptable salt thereof.

62. The pharmaceutical composition of any one of claims 58-61, in an aerosol, an inhalable powder, an injectable, a liquid, a solid, a capsule or a tablet form.

63. The pharmaceutical composition of claim 62, wherein the tablets are coated with a coating which inhibits oxygen from contacting the core.

64. The pharmaceutical composition of claim 63, wherein the coating comprises a cellulosic polymer, a detackifier, a gloss enhancer, or pigment.

65. The pharmaceutical composition of any one of claims 58-64, further comprising mannitol, an alkalinizing agent, an oxidation reducing agent, a lubricant, and/or a filler.

66. The pharmaceutical composition of claim 65, wherein the alkalinizing agent is meglumine.

67. The pharmaceutical composition of any one of claims 58-65, which is free of an alkalinizing agent or an oxidation reducing agent, preferably the pharmaceutical composition is free of an alkalinizing agent and free of an oxidation reducing agent.

68. The pharmaceutical composition of any one of claims 58-67, which is stable and free of disintegrant.

69. The pharmaceutical composition of claim 65, wherein the lubricant is present in the composition as solid particles.

70. The pharmaceutical composition of claim 65 or 69, wherein the lubricant is sodium stearyl fumarate or magnesium stearate.

71. The pharmaceutical composition of claim 65, wherein the filler is present in the composition as solid particles.

72. The pharmaceutical composition of claim 65 or 71, wherein the filler is lactose, lactose monohydrate, starch, isomalt, mannitol, sodium starch glycolate, sorbitol, lactose spray dried, lactose anhydrouse, or a combination thereof.

73. The pharmaceutical composition of any one of claims 58-72, wherein the amount of laquinimod in the composition is less than 0.6 mg, 0.03-600 mg, 0.1-40.0 mg, 0.1-2.5 mg, 0.25-2.0 mg, 0.5-1.2 mg, 0.25 mg, 0.3 mg, 0.5 mg, 0.6 mg, 1.0 mg, 1.2 mg, 1.5 mg, or 2.0 mg.

74. The pharmaceutical composition of any one of claims 58-73, wherein the amount of the compound of formula (I) is 12-7200 mg, 120 mg, 240 mg, 480 mg, or 720 mg.

75. A pharmaceutical composition in unit dosage form, useful in treating a subject afflicted with MS or presenting a CIS, which comprises:
a) an amount of laquinimod or a pharmaceutically acceptable salt thereof;
b) an amount of a compound of formula (I): 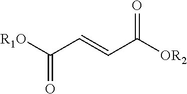
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, or a pharmaceutically acceptable salt thereof,
wherein the respective amounts of said laquinimod and said compound of formula (I) in said composition are effective, upon concomitant administration to said subject of one or more of said unit dosage forms of said composition, to treat the subject.

76. The pharmaceutical composition of claim 75, wherein the respective amounts of said laquinimod and said compound of formula (I) in said unit dose when taken together is more effective to treat the subject than when compared to the administration of said laquinimod in the absence of said compound of formula (I) or the administration of said compound of formula (I) in the absence of said laquinimod.

77. A pharmaceutical composition comprising an amount of laquinimod or a pharmaceutically acceptable salt thereof for use in treating a subject afflicted with MS or presenting a CIS as an add-on therapy or in combination with, or simultaneously, contemporaneously or concomitantly with a compound of formula (I): 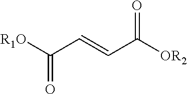
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, or a pharmaceutically acceptable salt thereof.

78. A pharmaceutical composition comprising an amount of a compound of formula (I): 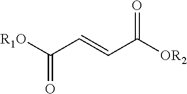
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, or a pharmaceutically acceptable salt thereof,
for use treating a subject afflicted with MS or presenting a CIS as an add-on therapy or in combination with, or simultaneously, contemporaneously or concomitantly with laquinimod or a pharmaceutically acceptable salt thereof.

79. Use of:
a) an amount of laquinimod or pharmaceutically acceptable salt thereof; and
b) an amount of a compound of formula (I): 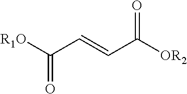
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, or pharmaceutically acceptable salt thereof,
in the preparation of a combination for treating a subject afflicted with MS or presenting a CIS wherein the amount of laquinimod and the amount of the compound of formula (I) are administered simultaneously or contemporaneously.

80. Laquinimod for use as an add-on therapy or in combination with a compound of formula (I): 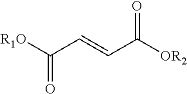
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, or a pharmaceutically acceptable salt thereof, in treating a subject afflicted with MS or presenting a CIS.

81. A compound of formula (I): 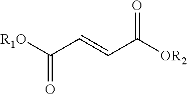
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, or a pharmaceutically acceptable salt thereof,
for use as an add-on therapy or in combination with laquinimod or a pharmaceutically acceptable salt thereof in treating a subject afflicted with MS or presenting a CIS.

82. Laquinimod or a pharmaceutically acceptable salt thereof and a compound of formula (I): 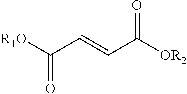
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, or a pharmaceutically acceptable salt thereof,
for the treatment of a subject afflicted with MS or presenting a CIS, wherein the laquinimod and the compound of formula (I) are administered simultaneously, separately or sequentially.

83. A product containing an amount of laquinimod or a pharmaceutically acceptable salt thereof and an amount of a compound of formula (I): 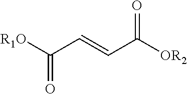
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, and, or a pharmaceutically acceptable salt thereof,
for simultaneous, separate or sequential use in treating a subject afflicted with MS or presenting a CIS.

84. A therapeutic package for dispensing to, or for use in dispensing to, a subject afflicted with MS or presenting a CIS, which comprises:
a) one or more unit doses, each such unit dose comprising:
i) an amount of laquinimod or a pharmaceutically acceptable salt thereof and
ii) an amount a compound of formula (I): 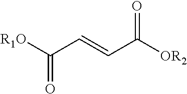
wherein
R₁ is H, C₁-C₁₂ alkyl, C₂-C₁₂ alkenyl, C₂-C₁₂ alkynyl or C₃-C₈ cycloalkyl; and
R₂ is H, C₁-C₁₂ alkyl, C₂-C₁₂ alkenyl, C₂-C₁₂ alkynyl or C₃-C₈ cycloalkyl; and
when one of R₁ or R₂ is CH₃, then the other of R₁ or R₂ is other than CH₃, or a pharmaceutically acceptable salt thereof
wherein the respective amounts of said laquinimod and said compound of formula (I) in said unit dose are effective, upon concomitant administration to said subject, to treat the subject, and
b) a finished pharmaceutical container therefor, said container containing said unit dose or unit doses, said container further containing or comprising labeling directing the use of said package in the treatment of said subject.

85. The therapeutic package of claim 84, wherein the respective amounts of said laquinimod and said compound of formula (I) in said unit dose when taken together is more effective to treat the subject than when compared to the administration of said laquinimod in the absence of said compound of formula (I) or the administration of said compound of formula (I) in the absence of said laquinimod.

86. The method of any one of claims 1-31, the package of any one of claims 32-57, the pharmaceutical composition of any one of claims 58-78, the use of any one of claims 79-83 or the therapeutic package of claim 84 or 85, wherein in the compound of formula (I):
R₁ is H, C₂-C₁₂ alkyl, C₂-C₁₂ alkenyl, C₂-C₁₂ alkynyl or C₃-C₈ cycloalkyl; and
R₂ is H, C₂-C₁₂ alkyl, C₂-C₁₂ alkenyl, C₂-C₁₂ alkynyl or C₃-C₈ cycloalkyl, or
wherein in the compound of formula (I):
R₁ is H, C₃-C₁₂ alkyl, C₂-C₁₂ alkenyl, C₂-C₁₂ alkynyl or C₃-C₈ cycloalkyl; and
R₂ is H, C₃-C₁₂ alkyl, C₂-C₁₂ alkenyl, C₂-C₁₂ alkynyl; or C₃-C₈ cycloalkyl.

87. The method, package, pharmaceutical composition, use or therapeutic package of claim 86, wherein in the compound of formula (I), when one of R₁ or R₂ is H, then the other of R₁ or R₂ is other than H, or a pharmaceutically acceptable salt thereof.

88. The method, package, pharmaceutical composition, use or therapeutic package of claim 86 or 87, wherein in the compound of formula (I), R₁ and R₂ are the same or R₁ and R₂ are different.

89. The method, package, pharmaceutical composition, use or therapeutic package of claim 86, wherein in the compound of formula (I):
R₁ is H; and R₂ is H;
R₁ is H; and R₂ is CH₂CH₃;
R₁ is H; and R₂ is CH₂CH₂CH₃;
R₁ is H; and R₂ is CH₂CH₂CH₂CH₃;
R₁ is H; and R₂ is CH₂CH₂CH₂CH₂CH₃;
R₁ is H; and R₂ is CH₂CH₂CH₂CH₂CH₂CH₃;
R₁ is CH₂CH₃; and R₂ is CH₂CH₃;
R₁ is CH₂CH₃; and R₂ is CH₂CH₂CH₃;
R₁ is CH₂CH₃; and R₂ is CH₂CH₂CH₂CH₃;
R₁ is CH₂CH₃; and R₂ is CH₂CH₂CH₂CH₂CH₃;
R₁ is CH₂CH₃; and R₂ is CH₂CH₂CH₂CH₂CH₂CH₃;
R₁ is CH₂CH₂CH₃; and R₂ is CH₂CH₂CH₃;
R₁ is CH₂CH₂CH₃; and R₂ is CH₂CH₂CH₂CH₃;
R₁ is CH₂CH₂CH₃; and R₂ is CH₂CH₂CH₂CH₂CH₃; or
R₁ is CH₂CH₂CH₃; and R₂ is CH₂CH₂CH₂CH₂CH₂CH₃.

90. The method of any one of claims 1-31, package of any one of claims 32-57, pharmaceutical composition of any one of claims 58-78, use of any one of claims 79-83 or therapeutic package of claim 84 or 85, wherein in the compound of formula (I), R₁ is H and R₂ is CH₃, or R₁ is H and R₂ is CH₂CH₃, or R₁ is CH₃ and R₂ is CH₂CH₃.

91. The method, package, pharmaceutical composition, use or therapeutic package of claim 86, wherein the compound of formula (I) is formoterol fumarate.
